(19) United States
(12) Patent Application Publication
(10) Pub. No.: US 2021/0393651 A1
(43) Pub. Date: Dec. 23, 2021

(54) TARGETING THE ONCOGENIC TRANSCRIPTION FACTOR STAT5 WITH MINERALOCORTICOID ANALOGUES

(71) Applicant: DANA-FARBER CANCER INSTITUTE, INC., Boston, MA (US)

(72) Inventors: David Frank, Lexington, MA (US); Erik Nelson, Cos Cob, CT (US)

(73) Assignee: DANA-FARBER CANCER INSTITUTE, INC., Boston, MA (US)

(21) Appl. No.: 17/289,162

(22) PCT Filed: Nov. 7, 2019
(86) PCT No.: PCT/US19/60251

§ 371 Date: Apr. 27, 2021

Related U.S. Application Data
(60) Provisional application No. 62/757,957, filed on Nov. 9, 2018.

(51) Int. Cl. A61K 31/573; A61K 45/06; A61P 35/00

(52) U.S. Cl. CPC A61K 31/573; A61P 35/00; A61K 45/06

(57) ABSTRACT

The present invention relates to compositions and methods for treating cancer with targeted mineralocorticoid analogues

### RELATED APPLICATIONS

This application claims the benefit of priority under 35 U.S.C. § 119(e) to U.S. Provisional Application No. 62/757,957, filed Nov. 9, 2018, which is incorporated herein by reference in its entirety.

### GOVERNMENT LICENSE RIGHTS

This invention was made with government support under grant number R01-CA160979 awarded by the National Institutes of Health. The government has certain rights in the invention.

### BACKGROUND OF THE INVENTION

The transcription factor, signal transducer and activator of transcription 5 (STAT5) regulates the expression of genes controlling the survival, proliferation, and differentiation of mammary epithelial cells. Under physiological conditions, STAT5 is activated rapidly and transiently to maintain tight control on the expression of target genes. However, many tumor types depend upon constitutive activation of STAT5, thereby driving expression of genes promoting malignant cellular behavior. Thus, prior to the invention described herein, there was a pressing need to develop inhibitors of STAT5 activity.

### SUMMARY OF THE INVENTION

The present invention is based upon the surprising discovery that mineralocorticoid receptor agonists and mineralocorticoid receptor antagonists unexpectedly inhibit signal transducer and activator of transcription 5 (STAT5).

Methods of inhibiting STAT5 function or activity in a cell are carried out by contacting the cell with a mineralocorticoid receptor agonist or a mineralocorticoid receptor antagonist, or an analogue thereof, thereby inhibiting STAT5 function or activity in a cell. For example, the mineralocorticoid receptor agonist comprises aldosterone, fludrocortisone, desoxycortone, hydrocortisone, methylprednisolone, prednisolone, prednisone, or an analogue thereof. Specifically, exemplary mineralocorticoid receptor agonists comprise fludrocortisone (fludrocortisone acetate), desoxycortone (desoxycorticosterone) (desoxycortone esters), hydrocortisone (cortisol) (hydrocortisone esters), methylprednisolone (methylprednisolone esters), prednisolone (prednisolone esters), and prednisone (prednisone esters). In another example, the mineralocorticoid receptor antagonist comprises spironolactone, canrenoate potassium, canrenone, drospirenone, dydrogesterone, eplerenone, gestodene, medrogestone, progesterone, trimegestone, amlodipine, aspararenone, benidipine, esaxerenone, felodipine, finerenone, nifedipine, nimodipine, nitrendipine, or an analogue thereof. Specifically, an exemplary mineralocorticoid receptor antagonists also comprises canrenoate potassium (potassium canrenoate).

For example, the STAT5 function or activity comprises STAT5-dependent gene expression/transcriptional activity. Other exemplary STAT5 function or activity includes STAT5 phosphorylation, STAT5 dimerization, STAT5 binding to a polynucleotide comprising a STAT5 binding site, STAT5 binding to genomic deoxyribonucleic acid (DNA), activation of a STAT5 responsive gene, and STAT5 nuclear translocation. In one aspect, the STAT5 function or activity in the cell is inhibited by 10%400%, e.g., 15%, 20%, 25%, 30%, 35%, 40%, 45%, 50%, 55%, 60%, 65%, 70%, 75%, 80%, 85%, 90%, 95%, or 100%.

In one aspect, the mineralocorticoid receptor agonist or the mineralocorticoid receptor antagonist inhibits expression of a STAT5 target gene selected from the group consisting of amphiregulin (AREG) and cytokine inducible SH2-containing protein (CISH), B-cell lymphoma 2 (Bcl-2), B-cell lymphoma-extra large (Bcl-x1), suppressor of cytokine signaling 1 (SOCS1), SOCS3, oncostatin-M, mitogen-activated protein (MAP) kinase phosphatase-1 (MKP-1), Pim-1, Pim-2, p21 (CIP/WAF1), interleukin-2 receptor a (IL-2Ra), IL-2Rβ, related to receptor tyrosine kinase (Ryk), and tumor necrosis factor receptor superfamily member 13B (TNFRSF13b).

In some cases, the mineralocorticoid receptor agonist or the mineralocorticoid receptor antagonist is administered at a dose of 0.01 μM to 10 μM, e.g., 0.05 μM, 0.10 μM, 0.20 μM, 0.30 μM, 0.40 μM, 0.50 μM, 0.60 μM, 0.70 μM, 0.80 μM, 0.90 μM, 1.0 μM, 1.5 μM, 2.0 μM, 2.5 μM, 3 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, 10 μM. For example, in some cases, fludrocortisone is administered at a dose of 0.1 μM.

Also provided are methods for treating or preventing a hyperproliferative disorder or an inflammatory disease associated with aberrant STAT5 activity in a subject, e.g., a human subject, by administering to the subject a therapeutically effective amount of a mineralocorticoid receptor agonist or a mineralocorticoid receptor antagonist, or an analogue thereof, thereby treating or preventing the hyperproliferative disorder or inflammatory disease associated with aberrant STAT5 activity in the subject. Exemplary modes of administration of the mineralocorticoid receptor agonist or a mineralocorticoid receptor antagonist include parental administration (e.g., subcutaneous and intravenous administration) and oral administration.

In some cases, the subject has been diagnosed with a hyperproliferative disorder or an inflammatory disease associated with aberrant STAT5 activity. In other cases, the subject is identified as having elevated STAT5 activity, or the subject is identified as in need of inhibiting STAT5 activity. For example, STAT5 activity in the subject is 5% elevated, 10% elevated, 20% elevated, 30% elevated, 40% elevated, 50% elevated, 60% elevated, 70% elevated, 80% elevated, 90% elevated, or 100% elevated. The subject in need of inhibition of STAT5 will generally display enhanced STAT5 activity as described herein. It is readily apparent to one of ordinary skill in the art, based on the teachings herein, how to determine whether an individual has enhanced STAT5 activity.

In one example, a mineralocorticoid receptor agonist or a mineralocorticoid receptor antagonist is administered soon after diagnosis with a hyperproliferative disorder, e.g., neoplasia, and before relapse of the disorder.

In some cases, a mineralocorticoid receptor agonist and a mineralocorticoid receptor antagonist are administered in combination. In other cases, one, two, three, or more mineralocorticoid receptor agonist(s) and/or one, two, three, or more mineralocorticoid receptor antagonist(s) are administered.

For example, the mineralocorticoid receptor agonist comprises aldosterone, fludrocortisone, desoxycortone, hydrocortisone, methylprednisolone, prednisolone, prednisone, or an analogue thereof. Specifically, exemplary mineralocorticoid receptor agonists include fludrocortisone (fludrocortisone acetate), desoxycortone (desoxycorticosterone) (desoxycortone esters), hydrocortisone (cortisol) (hydrocortisone esters), methylprednisolone (methylprednisolone esters), prednisolone (prednisolone esters), and prednisone (prednisone esters). In another example, the mineralocorticoid receptor antagonist comprises spironolactone, canrenoate potassium, canrenone, drospirenone, dydrogesterone, eplerenone, gestodene, medrogestone, progesterone, trimegestone, amlodipine, aspararenone, benidipine, esaxerenone, felodipine, finerenone, nifedipine, nimodipine, nitrendipine, or an analogue thereof. Specifically, an exemplary mineralocorticoid receptor antagonist also comprises canrenoate potassium (potassium canrenoate).

In one aspect, the STAT5 function or activity comprises STAT5-dependent gene expression/transcriptional activity. Other exemplary STAT5 function or activity includes STAT5 phosphorylation, STAT5 dimerization, STAT5 binding to a polynucleotide comprising a STAT5 binding site, STAT5 binding to genomic deoxyribonucleic acid (DNA), activation of a STAT5 responsive gene and STAT5 nuclear translocation.

For example, the mineralocorticoid receptor agonist or the mineralocorticoid receptor antagonist inhibits expression of a STAT5 target gene selected from the group consisting of AREG and CISH, Bcl-2, Bcl-x1, SOCS1, SOCS3, oncostatin-M, MKP-1, Pim-1, Pim-2, p21 (CIP/WAF1), IL-2Rα, IL-2Rβ, Ryk, and TNFRSF13b.

In some cases, the mineralocorticoid receptor agonist or the mineralocorticoid receptor antagonist is administered at a dose of 0.01 μM to 10 μM, e.g., 0.05 μM, 0.10 μM, 0.20 μM, 0.30 μM, 0.40 μM, 0.50 μM, 0.60 μM, 0.70 μM, 0.80 μM, 0.90 μM, 1.0 μM, 1.5 μM, 2.0 μM, 2.5 μM, 3 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, 10 μM. For example, fludrocortisone is administered at a dose of 0.1 μM.

In another example, the mineralocorticoid receptor agonist or the mineralocorticoid receptor antagonist is administered at a dose of about 50 mg to about 100 mg (e.g., about 50 mg, about 60 mg, about 70 mg, about 80 mg, about 90 mg, or about 100 mg) by mouth at least once daily (e.g., once daily, twice daily, three times daily or four times daily). In another example, the mineralocorticoid receptor agonist or the mineralocorticoid receptor antagonist is administered at a dose of about 1500 mg by mouth at least once daily (e.g., once daily, twice daily, three times daily or four times daily).

In one example, the mineralocorticoid receptor agonist or the mineralocorticoid receptor antagonist is administered once per month, once per week, once per day, every 12 hours, every 6 hours, every 4 hours, or every hour.

An exemplary hyperproliferative disorder comprises cancer. For example, the cancer comprises a solid tumor or a hematological cancer. Suitable solid tumors are selected from the group consisting of breast cancer, melanoma, colon cancer, ovarian cancer, pancreatic cancer, lung cancer, hepatic cancer, head and neck cancer, prostate cancer and brain cancer. In some cases, the hematological cancer comprises leukemia or multiple myeloma. For example, the leukemia is selected from the group consisting of acute lymphoblastic leukemia, acute myelogenous leukemia, chronic myelogenous leukemia, Hodgkin's disease, non-Hodgkin's lymphoma, T-cell lymphoma, B-cell lymphoma and chronic lymphocytic leukemia.

In some cases, the mineralocorticoid receptor agonist or the mineralocorticoid receptor antagonist inhibits or reduces the size of the cancer. For example, the mineralocorticoid receptor agonist or the mineralocorticoid receptor antagonist inhibits or reduces the size of a tumor by 5%, 10%, 15%, 20%, 25%, 30%, 35%, 40%, 45%, 50%, 55%, 60%, 65%, 70%, 75%, 80%, 85%, 90%, 95%, or 100%.

For example, the inflammatory disease associated with aberrant STAT5 activity comprises systemic lupus erythematosus, multiple sclerosis, Crohn's disease, ulcerative colitis, or graft versus host disease.

In some cases, the methods further comprise administering a chemotherapeutic agent selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and vinorelbine.

Also provided is a cell-based system that allows for the quantitative high-throughput measurement of STAT5-dependent gene expression. Specifically, provided is one or more isolated breast cancer cells, e.g., a T47D cell(s), wherein each cell comprises a vector (e.g., a plasmid) expressing a firefly luciferase reporter gene operably-linked (i.e., under the control of) a STAT5-dependent promoter.

Also provided is one or more isolated breast cancer cells, e.g., a T47D cell(s), wherein each cell comprises a vector, e.g., a plasmid, expressing a firefly luciferase reporter gene operably linked to a STAT5-dependent promoter sequence derived from a neural cell adhesion molecule 2 (NCAM2) gene (e.g., NCAM-luc or NCAM-luciferase) or region B of a B-cell lymphoma 6 (BCL6) gene (e.g., B-luc or B-luciferase). In some cases, the cells also comprise a vector, e.g., a plasmid, expressing Renilla luciferase under a constitutive promoter (e.g., pRL-TK). In one example, the cells also comprise a vector, e.g., a plasmid, expressing mineralocorticoid receptor (MCR) (e.g., pCMX-MCR).

Methods of screening for a compound that inhibits STAT5 function and/or activity are carried out by providing one or more breast cancer cell(s) comprising a vector expressing a firefly luciferase reporter gene operably-linked to a STAT5-dependent promoter, and contacting the cell(s) with a candidate compound, wherein a decrease in the level of STAT5-dependent luciferase activity in the presence of the candidate compound as compared to the level of STAT5-dependent luciferase activity in the absence of the candidate compound indicates that the candidate compound inhibits STAT5 function and/or activity.

In some cases, the methods further comprise contacting the cell with an agent that induces the function and/or activity of STAT5 prior to contacting the cell with a candidate compound. For example, the agent that induces the function and/or activity of STAT5 comprises prolactin.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “antineoplastic agent” is used herein to refer to agents that have the functional property of inhibiting a development or progression of a neoplasm in a human. Inhibition of metastasis is frequently a property of antineoplastic agents.

By “agent” is meant any small compound, antibody, nucleic acid molecule, or polypeptide, or fragments thereof.

By “agonist” is meant an agent capable of initiating the same reaction or activity typically produced by an endogenous substance. For example, an agonist binds to a receptor on a cell to initiate the same reaction or activity typically produced by the binding of the endogenous ligand.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by sodium dodecyl sulphate-polyacrylamide gel electrophoresis (SDS-PAGE) under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist” is used in the broadest sense, and includes an agent that partially or fully blocks, inhibits, or neutralizes a biological activity of an epitope, polypeptide, or cell that it specifically binds. Methods for identifying antagonists may comprise contacting a polypeptide or cell specifically bound by a candidate antagonist with the candidate antagonist and measuring a detectable change in one or more biological activities normally associated with the polypeptide or cell.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., cancer, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion, e.g., a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic complementary deoxyribonucleic acid (cDNA), a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by high performance liquid chromatography (HPLC) analysis.

By “immunogenicity” is meant the ability of a particular substance, such as an antigen or epitope, to provoke an immune response in the body of a human or animal.

By “marker” is meant any protein or polynucleotide having an alteration in expression level or activity that is associated with a disease or disorder, e.g., neoplasia.

By “modulate” is meant alter (increase or decrease). Such alterations are detected by standard art-known methods such as those described herein.

The term, “normal amount” refers to a normal amount of a complex in an individual known not to be diagnosed with neoplasia. The amount of the molecule can be measured in a test sample and compared to the “normal control level,” utilizing techniques such as reference limits, discrimination limits, or risk defining thresholds to define cutoff points and abnormal values (e.g., for neoplasia). The “normal control level” means the level of one or more proteins (or nucleic acids) or combined protein indices (or combined nucleic acid indices) typically found in a subject known not to be suffering from neoplasia. Such normal control levels and cutoff points may vary based on whether a molecule is used alone or in a formula combining other proteins into an index. Alternatively, the normal control level can be a database of protein patterns from previously tested subjects who did not convert to neoplasia over a clinically relevant time horizon. In another aspect, the normal control level can be a level relative to a housekeeping gene.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity, e.g., at least 80%, at least 85%, at least 90%, at least 95%, or at least 99% identity. Polynucleotides having “substantial identity” to an endogenous sequence are typically capable of hybridizing with at least one strand of a double-stranded nucleic acid molecule.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia and of its evolution as well as the prospect of recovery as anticipated from usual course of the disease. Once the aggressiveness has been determined, appropriate methods of treatments are chosen.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

The term “sample” as used herein refers to a biological sample obtained for the purpose of evaluation in vitro. Exemplary tissue samples for the methods described herein include tissue samples from tumors or the surrounding microenvironment (i.e., the stroma and/or infiltrating immune cells). With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukapheresis). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 70%, at least 80%, at least 85%, at least 90%, at least 95%, or at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “subject” as used herein includes all members of the animal kingdom prone to suffering from the indicated disorder. In some aspects, the subject is a mammal, e.g., a human mammal or a non-human mammal. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and National Center for Biotechnology Information (NCBI) submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1C is a series of bar charts showing that fludrocortisone specifically inhibits STAT5 transcriptional activity. FIG. 1A is a bar chart wherein reporter cell lines were treated with the indicated doses of fludrocortisone for two hours, after which cytokines were added to activate the appropriate transcription factor. Luciferase activity was quantified by luminometry six hours later. FIG. 1B is a bar chart wherein T47D cells were transfected with one of two luciferase constructs regulated by a STAT5-dependent promoter (NCAM-luc or B-luc) together with a normalizing control, and the effect of fludrocortisone on prolactin-induced gene expression was determined. Jak inhibitor 1 (JAKinhib; 1 μM) was used as a positive control. Error bars represent mean±SD for two independent experiments. FIG. 1C is a series of bar charts wherein the indicated breast cancer cell lines were pre-treated with vehicle (dimethylsulfoxide (DMSO)) or fludrocortisone (1 μM) for 1 hour, then left untreated or stimulated with prolactin (100 ng/ml) to induce STAT5 activation. RNA was harvested 1.5 hours later, and expression of the indicated STAT5 target genes was measured using quantitative reverse transcription polymerase chain reaction (RT-PCR) and normalized to the expression of 18s. Error bars represent mean±SD for triplicates.

FIG. 2A-FIG. 2E is a series of bar graphs showing that the endogenous mineralocorticoid aldosterone inhibits STAT5 transcriptional function. FIG. 2A is a bar chart wherein T47D cells were transfected with an MCR-dependent luciferase reporter and a normalizing control, and normalized luciferase activity was determined after the indicated treatment. FIG. 2B is a bar chart wherein T47D cells transfected with an MCR-dependent luciferase reporter and a normalizing control were treated with the indicated dose of aldosterone, after which luciferase activity was determined. FIG. 2C is a bar chart wherein T47D-luc cells were pre-treated with the indicated concentration of aldosterone for 1 hour, then stimulated with prolactin (100 ng/ml) for 6 hours, after which luciferase activity was determined. FIG. 2D is a bar chart wherein T47D cells were pre-treated with vehicle control or aldosterone (0.01 μM) for 1 hour, after which prolactin (100 ng/ml) was added to induce STAT5 activation. RNA was harvested 1.5 hours later, and expression of STAT5 target genes was measured using quantitative RT-PCR and normalized to the expression of 18s RNA. FIG. 2E is a bar chart wherein T47D cells stably expressing a STAT5-dependent luciferase reporter gene were treated with 1 μM fludrocortisone at the indicated time points relative to prolactin stimulation. Luciferase activity was quantified by luminometry six hours following prolactin stimulation. Error bars represent mean±SD for triplicates.

FIG. 3A-FIG. 3C is a series of photographs of blots and bar charts showing that fludrocortisone inhibits STAT5 transcriptional function without affecting its phosphorylation or genome binding. FIG. 3A is a photograph of a blot wherein T47D cells were pretreated with vehicle control or fludrocortisone for 1 hour after which prolactin was added. Cells were harvested at the indicated time point and immunoblotting was performed. FIG. 3B is a photograph of a blot wherein T47D cells were pretreated with vehicle, fludrocortisone, or Jak inhibitor 1 for 1 hour after which prolactin was added. Cells were collected 15 minutes after prolactin stimulation and cellular fractionation and immunoblotting was performed. FIG. 3C is a photograph of a blot wherein T47D cells were pretreated with vehicle (DMSO) or fludrocortisone for 1 hour, then left untreated or stimulated with prolactin. 30 minutes later, cells were harvested and ChIP was performed to analyze STAT5 and pol II binding to the CISH promoter region. Error bars represent mean±SD for two independent experiments.

FIG. 4A-FIG. 4C is a series of bar charts showing that the MCR cooperates with STAT5-mediated transcriptional induction. FIG. 4A is a bar chart wherein MCR mRNA was determined in the indicated breast cancer cell line by quantitative RT-PCR (normalized to 18S RNA). Error bars represent mean±SD for experiments performed in triplicate. FIG. 4B is a bar chart wherein STAT5-dependent luciferase activity was determined following prolactin stimulation in the indicated breast cancer cell lines. Error bars represent mean±SD for at least two independent experiments. FIG. 4C is a bar chart wherein expression of the STAT5 target gene STAT5 target gene CISH was determined by quantitative RT-PCR in breast cancer cells stimulated with prolactin for 90 minutes. FIG. 4D is a bar chart and a photograph of a blot wherein, Top, T47D-luc cells were transfected with siRNA targeting MCR or control, and 72 hours later prolactin was added to activate STAT5. Luciferase activity was quantitated by luminometry 6 hours later. Error bars represent mean±SD of three independent experiments. In FIG. 4D, Bottom, immunoblot analysis was performed to measure MCR expression after siRNA knock-down in T47D cells. Actin served as a loading control. FIG. 4E is a photograph of a blot wherein SKBR3 cells were transfected with an expression vector for MCR or control, and immunoblots were performed with the indicated antibodies. FIG. 4F is a bar chart wherein SKBR3 cells were transfected with a plasmid containing STAT5-dependent Ncam-luciferase (and a Renilla luciferase control), and an MCR expression plasmid (or vector control). Forty-eight hours later, prolactin was added to activate STAT5, and then 6 hours later a dual luciferase assay was performed. Error bars represent mean±SD for duplicate experiments. FIG. 4G is a series of bar charts wherein SKBR3 cells transfected with an MCR expression plasmid or a vector control were stimulated with prolactin (100 ng/ml) to activate STAT5, and RNA was harvested 1.5 hours later. Expression of the indicated STAT5 target genes was measured using quantitative RT-PCR and normalized to 18s RNA. Error bars represent mean±SD of two independent experiments.

### DETAILED DESCRIPTION OF THE INVENTION

The invention is based, at least in part, upon the identification that mineralocorticoid receptor agonists (e.g., fludrocortisone) and mineralocorticoid receptor antagonists (e.g., spironolactone) inhibit STAT5-dependent transcription/gene expression. Specifically, fludrocortisone inhibits STAT5-dependent gene expression in breast cancer cells by blocking the co-activating effects of the mineralocorticoid receptor.

As described herein, the transcription factor, STAT5, regulates the expression of genes controlling the survival, proliferation, and differentiation of mammary epithelial cells. Under physiological conditions, STAT5 is activated rapidly and transiently to maintain tight control on the expression of target genes. However, in breast cancer cells, STAT5 becomes activated constitutively, thereby driving expression of genes promoting malignant cellular behavior. As described herein, to gain further insight into the mechanisms of STAT5-mediated gene expression, a chemical library was screened to identify modulators of STAT5 transcriptional function. From this approach, the synthetic mineralocorticoid, fludrocortisone, was identified as a potent and specific STAT5 inhibitor. Both fludrocortisone and the endogenous mineralocorticoid, aldosterone, inhibited STAT5-dependent gene expression in breast cancer cells, without altering the activating phosphorylation, nuclear localization, or genomic binding of STAT5. Depletion of the MCR by RNA interference decreased STAT5-dependent gene expression. Conversely, expression of the MCR in breast cancer cells with low endogenous MCR expression enhanced STAT5-dependent gene expression. These findings presented in the Examples herein reveal a functional cooperativity between the MCR and STAT5, and suggest MCR serves as an important cofactor that modulates STAT5 transcriptional function in breast cancer and normal epithelial cells.

### Signal Transducers and Activators of Transcription (STAT) Molecules

Signal transducers and activators of transcription (STATs) are a family of transcription factors that play important roles in a range of cellular functions. STATs reside in the cytoplasm under basal conditions. Upon activation by tyrosine phosphorylation, STATs dimerize, translocate to the nucleus, bind to DNA, and regulate transcription of target genes that regulate cellular functions such as survival, proliferation, and differentiation (Darnell, J. E., Jr., 1997 Science 277, 1630-1635). Under physiological conditions, STATs are activated only transiently. By contrast, in many forms of cancer, STAT family members are activated constitutively and drive the expression of genes underlying malignant cellular behavior.

Specifically, members of the STAT protein family are intracellular transcription factors that mediate many aspects of cellular immunity, proliferation, apoptosis and differentiation. There are seven mammalian STAT family members that have been identified: STAT1, STAT2, STAT3, STAT4, STAT5 (STAT5A and STATSB), and STATE. STAT proteins are primarily activated by membrane receptor-associated Janus kinases (JAK). Dysregulation of the JAK/STAT pathway is frequently observed in primary tumors and leads to increased angiogenesis, enhanced survival of tumors, and immunosuppression. STAT proteins are involved in the development and function of the immune system and play a role in maintaining immune tolerance and tumor surveillance.

STAT proteins are present in the cytoplasm of cells under basal conditions. When activated by tyrosine phosphorylation, STAT proteins form dimers and translocate to the nucleus where they can bind specific nine-base-pair sequences in the regulatory regions of target genes, thereby activating transcription. A variety of tyrosine kinases, including polypeptide growth factor receptors, Src family members, and other kinases can catalyze this phosphorylation. While tyrosine phosphorylation is essential for their activation, STAT proteins can also be phosphorylated on unique serine residues. Although this is not sufficient to induce dimerization and DNA binding, STAT serine phosphorylation modulates the transcriptional response mediated by a tyrosine-phosphorylated STAT dimer, and may mediate distinct biological effects (Zhang X, et al. Science 1995; 267:1990-1994; Wen Z, et al. Cell 1995; 82:241-250; Kumar A, et al. Science 1997; 278:1630-1632.). STAT proteins function inappropriately in many human malignancies (Alvarez J V, et al., Cancer Res 2005; 65(12):5054-62; Frank D A, et al. Cancer Treat. Res. 2003; 115:267-291; Bowman T, et al. Oncogene 2000; 19(21):2474-88).

### Signal Transducer and Activator of Transcription 5 (STAT5)

STAT5 refers to two highly related proteins, STAT5A and STAT5B, which are part of the seven-membered STAT family of proteins. STAT5A and STAT5B are encoded by separate genes; however, the proteins are 90% identical. STAT5 is involved in cytosolic signalling and in mediating the expression of target genes, as described above. Aberrant STAT5 activity is linked to many cancer types.

Specifically, STAT5, which encompasses two highly homologous proteins, STAT5a and STAT5b, plays an important role in mammary gland development and in breast cancer. In the mammary gland, STAT5 is activated late in pregnancy in response to prolactin to promote terminal differentiation and milk production (Cui et al., 2004 Mol Cell Biol 24, 8037-8047; Iavnilovitch, et al., 2002 Mol Cancer Res 1, 32-47; Liu et al., 1995 Proc Natl Acad Sci USA 92, 8831-8835). In breast cancer, constitutively activated STAT5 enhances both survival and anchorage-independent growth of human mammary carcinoma cells (Tang et al., 2010 Endocrinology 151, 43-55). Mice that express a constitutively activated form of STAT5 develop mammary carcinomas, whereas mice that lack STAT5a are protected against mammary tumors induced by transforming growth factor α (Iavnilovitch, et al., 2002 Mol Cancer Res 1, 32-47; Ren et al., 2002 Oncogene 21, 4335-4339; Iavnilovitch et al., 2004 Int J Cancer 112, 607-619). Recently, it was found that Jak-mediated STAT5 signaling closely interacts with the PI3K/AKT pathway, and can mediate resistance to phosphoinositide 3-kinase/protein kinase B (PI3K/AKT) inhibition in breast cancer (Britschgi et al., 2012 Cancer cell 22, 796-811). Reflecting its role in promoting survival and proliferation of mammary epithelium, STAT5 is constitutively activated in a large fraction of human breast cancers (Walker et al., 2009 Mol Cancer Res 7, 966-976). The activation of STAT5 in breast cancer may be due to the autocrine, paracrine, or endocrine secretion of prolactin. In fact, the level of circulating prolactin correlates with risk of breast cancer in both premenopausal and postmenopausal women (Tworoger et al., 2006 Cancer Res 66, 2476-2482). Taken together, and as described in more detail below, these data suggest STAT5 plays an important role in breast cancer initiation and progression.

An exemplary human STATSA amino acid sequence is set forth below (SEQ ID NO: 11; GenBank Accession No: NP_001275647, Version 1, incorporated herein by reference):

| | |
|---|---|
| 1 | magwiqaqql qgdalrqmqv lygqhfpiev rhylaqwies qpwdaidldn pqdraqatql |
| 61 | leglvqelqk kaehqvgedg fllkiklghy atqlqktydr cplelvrcir hilyneqrlv |
| 121 | reanncsspa gilvdamsqk hlqinqtfee lrlvtqdten elkklqqtqe yfiiqyqesl |
| 181 | riqaqfaqla qlspqerlsr etalqqkqvs leawlqreaq tlqqyrvela ekhqktlqll |
| 241 | rkqqtiildd eliqwkrrqq lagnggppeg sldvlqswce klaeiiwqnr qqirraehlc |
| 301 | qqlpipgpve emlaevnati tdiisalvts tfiiekqppq vlktqtkfaa tvrllvggkl |
| 361 | nvhmnppqvk atiiseqqak sllknentrn ecsgeilnnc cvmeyhqatg tlsahfrnms |
| 421 | lkrikradrr gaesvteekf tvlfesqfsv gsnelvfqvk tlslpvvviv hgsqdhnata |
| 481 | tvlwdnafae pgrvpfavpd kvlwpqlcea lnmkfkaevq snrgltkenl vflaqklfnn |
| 541 | ssshledysg lsvswsqfnr enlpgwnytf wqwfdgvmev lkkhhkphwn dgailgfvnk |
| 601 | qqahdllink pdgtfllrfs dseiggitia wkfdspernl wnlkpfttrd fsirsladrl |
| 661 | gdlsyliyvf pdrpkdevfs kyytpvlaka vdgyvkpqik qvvpefvnas adaggssaty |
| 721 | mdqapspavc pqapynmypq npdhvldqdg efdldetmdv arhveellrr pmdsldsrls |
| 781 | ppaglftsar gsls |

An exemplary human STAT5A nucleic acid sequence is set forth below (SEQ ID NO: 12; GenBank Accession No: NM_001288718, Version 1, incorporated herein by reference):

| | |
|---|---|
| 1 | agatggccgg agtaaaagaa ggagggaggt gctgcggtgg tgggggtgat cttggcttca |
| 61 | ctagaatccc cagttcttcc cctctctaca gttttgtctc tgaggtcaca aaacctgtgg |
| 121 | cccccaagac acacatgcgc acacacgcgc gtgcacacac acaccccaca catttatttt |
| 181 | ttaatctagg ggctcaaaag atgacacgcg ccagagctgg aaggcgtcgc caattggtcc |
| 241 | acttttccct cctccctttt tgcggatgag aaaactgagg cccaggtttg ggatttccag |
| 301 | agcccgggat ttcccggcaa cgcccgacaa ccacattccc ccggctattc tgacccgccc |
| 361 | cggttccggg acgctccctg ggagccgccg ccgagggcct gctgggactc ccgggggacc |
| 421 | ccgccgtcgg ggcagccccc acgcccggcg ccgcccgccg ggaacggccg ccgctgttgc |
| 481 | gcacttgcag gggagccggc gactgagggc gaggcaggga gggagcaagc ggggctggga |
| 541 | gggctgctgg cgcgggctcg cgcgctgtgt atggtctatc gcaggcagct gacctttgag |
| 601 | gaggaaatcg ctgctctccg ctccttcctg tagtaacagc cgccgctgcc gccgccgcca |
| 661 | ggaaccccgg ccgggagcga gagccgcggg gcgcagagcc ggcccggctg ccggacggtg |
| 721 | cggccccacc aggtgaacgg ccatggcggg ctggatccag gcccagcagc tgcagggaga |
| 781 | cgcgctgcgc cagatgcagg tgctgtacgg ccagcacttc cccatcgagg tccggcacta |
| 841 | cttggcccag tggattgaga gccagccatg ggatgccatt gacttggaca atccccagga |
| 901 | cagagcccaa gccacccagc tcctggaggg cctggtgcag gagctgcaga agaaggcgga |
| 961 | gcaccaggtg ggggaagatg ggtttttact gaagatcaag ctggggcact acgccacgca |
| 1021 | gctccagaaa acatatgacc gctgccccct ggagctggtc cgctgcatcc ggcacattct |
| 1081 | gtacaatgaa cagaggctgg tccgagaagc caacaattgc agctctccgg ctgggatcct |
| 1141 | ggttgacgcc atgtcccaga agcaccttca gatcaaccag acatttgagg agctgcgact |
| 1201 | ggtcacgcag gacacagaga atgagctgaa gaaactgcag cagactcagg agtacttcat |
| 1261 | catccagtac caggagagcc tgaggatcca agctcagttt gcccagctgg cccagctgag |
| 1321 | cccccaggag cgtctgagcc gggagacggc cctccagcag aagcaggtgt ctctggaggc |
| 1381 | ctggttgcag cgtgaggcac agacactgca gcagtaccgc gtggagctgg ccgagaagca |
| 1441 | ccagaagacc ctgcagctgc tgcggaagca gcagaccatc atcctggatg acgagctgat |
| 1501 | ccagtggaag cggcggcagc agctggccgg gaacggcggg ccccccgagg gcagcctgga |
| 1561 | cgtgctacag tcctggtgtg agaagttggc cgagatcatc tggcagaacc ggcagcagat |
| 1621 | ccgcagggct gagcacctct gccagcagct gcccatcccc ggcccagtgg aggagatgct |
| 1681 | ggccgaggtc aacgccacca tcacggacat tatctcagcc ctggtgacca gcacattcat |
| 1741 | cattgagaag cagcctcctc aggtcctgaa gacccagacc aagtttgcag ccaccgtacg |
| 1801 | cctgctggtg ggcgggaagc tgaacgtgca catgaatccc ccccaggtga aggccaccat |
| 1861 | catcagtgag cagcaggcca agtctctgct taaaaatgag aacacccgca acgagtgcag |
| 1921 | tggtgagatc ctgaacaact gctgcgtgat ggagtaccac caagccacgg gcaccctcag |
| 1981 | tgcccacttc aggaacatgt cactgaagag gatcaagcgt gctgaccggc ggggtgcaga |
| 2041 | gtccgtgaca gaggagaagt tcacagtcct gtttgagtct cagttcagtg ttggcagcaa |
| 2101 | tgagcttgtg ttccaggtga agactctgtc cctacctgtg gttgtcatcg tccacggcag |
| 2161 | ccaggaccac aatgccacgg ctactgtgct gtgggacaat gcctttgctg agccgggcag |
| 2221 | ggtgccattt gccgtgcctg acaaagtgct gtggccgcag ctgtgtgagg cgctcaacat |
| 2281 | gaaattcaag gccgaagtgc agagcaaccg gggcctgacc aaggagaacc tcgtgttcct |
| 2341 | ggcgcagaaa ctgttcaaca acagcagcag ccacctggag gactacagtg gcctgtccgt |
| 2401 | gtcctggtcc cagttcaaca gggagaactt gccgggctgg aactacacct tctggcagtg |
| 2461 | gtttgacggg gtgatggagg tgttgaagaa gcaccacaag ccccactgga atgatggggc |
| 2521 | catcctaggt tttgtgaata agcaacaggc ccacgacctg ctcatcaaca agcccgacgg |
| 2581 | gaccttcttg ttgcgcttta gtgactcaga aatcgggggc atcaccatcg cctggaagtt |
| 2641 | tgactccccg gaacgcaacc tgtggaacct gaaaccattc accacgcggg atttctccat |
| 2701 | caggtccctg gctgaccggc tgggggacct gagctatctc atctatgtgt ttcctgaccg |
| 2761 | ccccaaggat gaggtcttct ccaagtacta cactcctgtg ctggctaaag ctgttgatgg |
| 2821 | atatgtgaaa ccacagatca agcaagtggt ccctgagttt gtgaatgcat ctgcagatgc |
| 2881 | tgggggcagc agcgccacgt acatggacca ggccccctcc ccagctgtgt gcccccaggc |
| 2941 | tccctataac atgtacccac agaaccctga ccatgtactc gatcaggatg gagaattcga |
| 3001 | cctggatgag accatggatg tggccaggca cgtggaggaa ctcttacgcc gaccaatgga |
| 3061 | cagtcttgac tcccgcctct cgccccctgc cggtcttttc acctctgcca gaggctccct |
| 3121 | ctcatgaatg tttgaatccc acgcttctct ttggaaacaa tatgcaatgt gaagcggtcg |
| 3181 | tgttgtgagt ttagtaaggc tgtgtacact gacacctttg caggcatgca tgtgcttgtg |
| 3241 | tgtgtgtgtg tgtgtgtgtc cttgtgcatg agctacgcct gcctcccctg tgcagtcctg |
| 3301 | ggatgtggct gcagcagcgg tggcctcttt tcagatcatg gcatccaaga gtgcgccgag |
| 3361 | tctgtctctg tcatggtaga gaccgagcct ctgtcactgc aggcactcaa tgcagccaga |
| 3421 | cctattcctc ctgggcccct catctgctca gcagctattt gaatgagatg attcagaagg |
| 3481 | ggaggggaga caggtaacgt ctgtaagctg aagtttcact ccggagtgag aagctttgcc |
| 3541 | ctcctaagag agagagacag agagacagag agagagaaag agagagtgtg tgggtctatg |
| 3601 | taaatgcatc tgtcctcatg tgttgatgta accgattcat ctctcagaag ggaggctggg |
| 3661 | gttcattttc gagtagtatt ttatacttta gtgaacgtgg actccagact ctctgtgaac |
| 3721 | cctatgagag cgcgtctggg cccggccatg tccttagcac aggggggccg ccggtttgag |
| 3781 | tgagggtttc tgagctgctc tgaattagtc cttgcttggc tgcttggcct tgggcttcat |
| 3841 | tcaagtctat gatgctgttg cccacgtttc ccgggatata tattctctcc cctccgttgg |
| 3901 | gccccagcct tctttgcttg cctctctgtt tgtaaccttg tcgacaaaga ggtagaaaag |
| 3961 | attgggtcta ggatatggtg ggtggacagg ggccccggga cttggagggt tggtcctctt |
| 4021 | gcctcctgga aaaaacaaaa acaaaaaact gcagtgaaag acaagctgca aatcagccat |
| 4081 | gtgctgcgtg cctgtggaat ctggagtgag gggtaaaagc tgatctggtt tgactccgct |
| 4141 | ggaggtgggg cctggagcag gccttgcgct gttgcgtaac tggctgtgtt ctggtgaggc |
| 4201 | cttgctccca accccacacg ctcctccctc tgaggctgta ggactcgcag tcaggggcag |
| 4261 | ctgaccatgg aagattgaga gcccaaggtt taaacttctc tgaagggagg tggggatgag |
| 4321 | aagaggggtt tttttgtact ttgtacaaag accacacatt tgtgtaaaca gtgttttgga |
| 4381 | ataaaatatt tttttcataa aaaaaaaaaa aaaa |

An exemplary human STAT5B amino acid sequence is set forth below (SEQ ID NO: 13; GenBank Accession No: NP_036580, Version 2, incorporated herein by reference):

| | |
|---|---|
| 1 | mavwiqaqql qgealhqmqa lygqhfpiev rhylsqwies qawdsvdldn pqenikatql |
| 61 | leglvqelqk kaehqvgedg fllkiklghy atqlqntydr cpmelvrcir hilyneqrlv |
| 121 | reanngsspa gsladamsqk hlqinqtfee lrlvtqdten elkklqqtqe yfiiqyqesl |
| 181 | riqaqfgpla qlspqerlsr etalqqkqvs leawlqreaq tlqqyrvela ekhqktlqll |
| 241 | rkqqtiildd eliqwkrrqq lagnggppeg sldvlqswce klaeiiwqnr qqirraehlc |
| 301 | qqlpipgpve emlaevnati tdiisalvts tfiiekqppq vlktqtkfaa tvrllvggkl |
| 361 | nvhmnppqvk atiiseqqak sllknentrn dysgeilnnc cvmeyhqatg tlsahfrnms |
| 421 | lkrikrsdrr gaesvteekf tilfesqfsv ggnelvfqvk tlslpvvviv hgsqdnnata |
| 481 | tvlwdnafae pgrvpfavpd kvlwpqlcea lnmkfkaevq snrgltkenl vflaqklfnn |
| 541 | ssshledysg lsvswsqfnr enlpgrnytf wqwfdgvmev lkkhlkphwn dgailgfvnk |
| 601 | qqahdllink pdgtfllrfs dseiggitia wkfdsqermf wnlmpfttrd fsirsladrl |
| 661 | gdlnyliyvf pdrpkdevys kyytpvpces atakavdgyv kpqikqvvpe fvnasadagg |
| 721 | gsatymdqap spavcpqahy nmypqnpdsv ldtdgdfdle dtmdvarrve ellgrpmdsq |
| 781 | wiphaqs |

An exemplary human STAT5B nucleic acid sequence is set forth below (SEQ ID NO: 14; GenBank Accession No: NM_012448, Version 3, incorporated herein by reference):

| | |
|---|---|
| 1 | ggcgggagga gagtcggcgg ccggagccgt caccccgggc ggggacccag cgcaggcaac |
| 61 | tccgcgcggc ggcccggccg agggagggag cgagcgggcg ggcgggcaag ccagacagct |
| 121 | gggccggagc agccgcgggc gcccgagggg ccgagcgaga ttgtaaacca tggctgtgtg |
| 181 | gatacaagct cagcagctcc aaggagaagc ccttcatcag atgcaagcgt tatatggcca |
| 241 | gcattttccc attgaggtgc ggcattattt atcccagtgg attgaaagcc aagcatggga |
| 301 | ctcagtagat cttgataatc cacaggagaa cattaaggcc acccagctcc tggagggcct |
| 361 | ggtgcaggag ctgcagaaga aggcagagca ccaggtgggg gaagatgggt ttttactgaa |
| 421 | gatcaagctg gggcactatg ccacacagct ccagaacacg tatgaccgct gccccatgga |
| 481 | gctggtccgc tgcatccgcc atatattgta caatgaacag aggttggtcc gagaagccaa |
| 541 | caatggtagc tctccagctg gaagccttgc tgatgccatg tcccagaaac acctccagat |
| 601 | caaccagacg tttgaggagc tgcgactggt cacgcaggac acagagaatg agttaaaaaa |
| 661 | gctgcagcag actcaggagt acttcatcat ccagtaccag gagagcctga ggatccaagc |
| 721 | tcagtttggc ccgctggccc agctgagccc ccaggagcgt ctgagccggg agacggccct |
| 781 | ccagcagaag caggtgtctc tggaggcctg gttgcagcgt gaggcacaga cactgcagca |
| 841 | gtaccgcgtg gagctggccg agaagcacca gaagaccctg cagctgctgc ggaagcagca |
| 901 | gaccatcatc ctggatgacg agctgatcca gtggaagcgg cggcagcagc tggccgggaa |
| 961 | cggcgggccc cccgagggca gcctggacgt gctacagtcc tggtgtgaga agttggccga |
| 1021 | gatcatctgg cagaaccggc agcagatccg cagggctgag cacctctgcc agcagctgcc |
| 1081 | catccccggc ccagtggagg agatgctggc cgaggtcaac gccaccatca cggacattat |
| 1141 | ctcagccctg gtgaccagca cgttcatcat tgagaagcag cctcctcagg tcctgaagac |
| 1201 | ccagaccaag tttgcagcca ctgtgcgcct gctggtgggc gggaagctga acgtgcacat |
| 1261 | gaaccccccc caggtgaagg ccaccatcat cagtgagcag caggccaagt ctctgctcaa |
| 1321 | gaacgagaac acccgcaatg attacagtgg cgagatcttg aacaactgct gcgtcatgga |
| 1381 | gtaccaccaa gccacaggca cccttagtgc ccacttcagg aatatgtccc tgaaacgaat |
| 1441 | taagaggtca gaccgtcgtg gggcagagtc ggtgacagaa gaaaaattta caatcctgtt |
| 1501 | tgaatcccag ttcagtgttg gtggaaatga gctggttttt caagtcaaga ccctgtccct |
| 1561 | gccagtggtg gtgatcgttc atggcagcca ggacaacaat gcgacggcca ctgttctctg |
| 1621 | ggacaatgct tttgcagagc ctggcagggt gccatttgcc gtgcctgaca aagtgctgtg |
| 1681 | gccacagctg tgtgaggcgc tcaacatgaa attcaaggcc gaagtgcaga gcaaccgggg |
| 1741 | cctgaccaag gagaacctcg tgttcctggc gcagaaactg ttcaacaaca gcagcagcca |
| 1801 | cctggaggac tacagtggcc tgtctgtgtc ctggtcccag ttcaacaggg agaatttacc |
| 1861 | aggacggaat tacactttct ggcaatggtt tgacggtgtg atggaagtgt taaaaaaaca |
| 1921 | tctcaagcct cattggaatg atggggccat tttggggttt gtaaacaagc aacaggccca |
| 1981 | tgacctactc attaacaagc cagatgggac cttcctcctg agattcagtg actcagaaat |
| 2041 | tggcggcatc accattgctt ggaagtttga ttctcaggaa agaatgtttt ggaatctgat |
| 2101 | gccttttacc accagagact tctccattcg gtccctagcc gaccgcttgg gagacttgaa |
| 2161 | ttaccttatc tacgtgtttc ctgatcggcc aaaagatgaa gtatactcca aatactacac |
| 2221 | accagttccc tgcgagtctg ctactgctaa agctgttgat ggatacgtga agccacagat |
| 2281 | caagcaagtg gtccctgagt ttgtgaacgc atctgcagat gccgggggcg gcagcgccac |
| 2341 | gtacatggac caggccccct ccccagctgt gtgtccccag gctcactata acatgtaccc |
| 2401 | acagaaccct gactcagtcc ttgacaccga tggggacttc gatctggagg acacaatgga |
| 2461 | cgtagcgcgg cgtgtggagg agctcctggg ccggccaatg gacagtcagt ggatcccgca |
| 2521 | cgcacaatcg tgaccccgcg acctctccat cttcagcttc ttcatcttca ccagaggaat |
| 2581 | cactcttgtg gatgttttaa ttccatgaat cgcttctctt ttgaaacaat actcataatg |
| 2641 | tgaagtgtta atactagttg tgaccttagt gtttctgtgc atggtggcac cagcgaaggg |
| 2701 | agtgcgagta tgtgtttgtg tgtgtgtgtg tgtgtgtgtg tgtgtgcgtg tttgcacgtt |
| 2761 | atggtgtttc tccctctcac tgtctgagag tttagttgta gcagaggggc cacagacaga |
| 2821 | agctgtggtg gtttttactt tgtgcaaaaa ggcagtgagt ttcgtgaagc ctggaagttg |
| 2881 | gccatgtgtc ttaagagtgg ctggactttg acatgtggct gtttgaataa gagaaggaca |
| 2941 | aagggaggag aaagcacatg tgctccagtg agtcttcgtc actctgtctg ccaagcaatt |
| 3001 | gatatataac cgtgattgtc tctgcttttc ttctgaaatg tagataactg ctttttgaca |
| 3061 | aagagagcct tccctctccc ccacccctgt gttcttgggt aggaatggga aaaggggcaa |
| 3121 | cctacaaaga ttgttggggc aagggaagtc acaagctttc ggatgggcgg tggcttttca |
| 3181 | caaaacattt agctcatctt attctctctt tgtcctctct cccctcctgc ccgcccgcac |
| 3241 | cctggaattg ccactcagtt cctctgggtg tgcacatatg tttggagaaa tagaggagag |
| 3301 | aaaagagggc cacgtaactg agagcttaca gtgccaatgc cgtttgtgtt ctggccagag |
| 3361 | tggagtgcgc agccctgact cccaggcgct gagattgttg cctggttacc caggaagctg |
| 3421 | ctgttccggc tgcccagcct ttctctgagc cagcggatgc acagtccgtg gccttcttca |
| 3481 | ggcttattga tgatgctttt tgcaaatgtt gaatcatggt tctgtttcta agttggatct |
| 3541 | tttttgtttt ctccttgcca ccctaatttg acatcaaaat tctctcttgt gcattgggcc |
| 3601 | ctgggtcatt caaacccagg tcacctcatt ccccttctct gttcacacct aatgtcttga |
| 3661 | agagtaggta gcagcagtgt gggctgaacc taggccagct tgcttagcgg gtcaccctgc |
| 3721 | tgtgaagtcc tggcaggtgt tggtaatgtg tggaaatgca gtcagcaagt ttgctgggga |
| 3781 | gtttgataaa agtataaaac aaaacaaaaa aagcctcggt ataattttgt tccacgactt |
| 3841 | cttctgtagc tttacaccag aaggaaggaa tgggctacag caggtagtgg aggaagaggg |
| 3901 | gggtgagcag gtgtattaaa atagcttacg ggtaaggcct aaaaggtcac ccctcggccc |
| 3961 | cctctccaaa agaagggcat gggcaccccc aggagaggat ggccccaaaa accttatttt |
| 4021 | tatacatgag agtaaataaa catatttttt ttacaaaaat aacttctgaa tttatcagtg |
| 4081 | ttttgccgtt aaaaatattc ctctatagta aattatttat tggaagatga cttttttaaa |
| 4141 | gctgccgttt gccttggctt ggtttcatac actgatttat ttttctatgc caggcagtag |
| 4201 | agtctctctg cctctgagga gcaggctacc cgcatcccac tcagcccctc cctacccctc |
| 4261 | aagatttgat gaaaattcca accatgagga tgggtgcatc ggggaagggt gagaaggaga |
| 4321 | gcctgcctgc tcagggatcc aggctcgtag agtcactccc tgcccgtctc ccagagatgc |
| 4381 | ttcaccagca cctgcctctg agacctcgct ctctgttcca gcaaccctgg ttggggggtc |
| 4441 | agacttgata cactttcagg ttgggagtgg acccacccca gggcctgctg aggacagagc |
| 4501 | agccaggccg tcctggctca ctttgcagtt ggcactgggt tggggaggaa gagagctgat |
| 4561 | gagtgtggct tccctgagct ggggtttccc tgcttgtcca gttgtgagct gtcctcggtg |
| 4621 | ttaccgaggc tgtgcctaga gagtggagat ttttgatgaa aggtgtgctc gctctctgcg |
| 4681 | ttctatcttc tctctcctcc ttgttcctgc aaaccacaag ataaaggtag tggtgtgtct |
| 4741 | cgaccccatc agcctctcac ccactcccag acacacacaa gtcctcaaaa gtttcagctc |
| 4801 | cgtgtgtgag atgtgcaggt tttttctagg gggtaggggg agactaaaat cgaatataac |
| 4861 | ttaaaatgaa agtatacttt ttataatttt tctttttaaa acttggtgaa attatttcag |
| 4921 | atacatattt tagtgtcaag gcagattagt tatttagcca ccaaaaaaaa gtattgtgta |
| 4981 | caatttgggg cctcaaattt gactctgcct caaaaaaaag aaatatatcc tatgcagagt |
| 5041 | tacagtcaca aagttgtgta ttttatgtta caataaagcc ttcctctgaa gggaaaaaaa |
| 5101 | aaaaaaaaaa aaaaaaaaaa aaaaaaaaaa aaaaaaaaaa aaaaaaaaaa aaaaaaaaaa |
| 5161 | aaaaaaaaaa a |

### Breast Cancer

Breast cancer is a type of cancer that develops in breast tissue. Signs of breast cancer include breast lumps, breast shape change, skin dimpling, fluid coming from the nipple, or a red/scaly patch of breast skin. Bone pain, swollen lymph nodes, shortness of breath, or yellow skin may be present in those with spread of the disease beyond the breast.

Risk factors for developing breast cancer include female gender, obesity, lack of physical exercise, drinking alcohol, hormone replacement therapy during menopause, ionizing radiation, early age at first menstruation, having children late or not at all, older age, and family history. For example, in some cases, genes inherited from a person's parents, including breast cancer type 1 susceptibility protein (BRCA1) and BRCA2, among others, contribute to disease. Breast cancer most commonly develops in cells from either (1) the lining of milk ducts (ductal carcinomas); or (2) the lobules that supply the ducts with milk (lobular carcinomas); however, there are more than 18 other sub-types of breast cancer.

The diagnosis of breast cancer is confirmed by taking a biopsy of the concerning lump. Breast cancer is often treated with platinum compounds, e.g., cisplatin, carboplatin or oxaliplatin, that cause inter-strand cross-links in DNA.

### Leukemia

Leukemia includes a group of cancers that usually begin in the bone marrow and result in high numbers of abnormal white blood cells called blasts or leukemia cells. Symptoms may include bleeding, bruising, feeling tired, fever, and an increased risk of infections. Risk factors for developing leukemia include smoking, ionizing radiation, some chemicals (e.g., benzene), prior chemotherapy, Down syndrome, and people with a family history of leukemia. There are four main types of leukemia: acute lymphoblastic leukemia (ALL), acute myeloid leukemia (AML), chronic lymphocytic leukemia (CLL) and chronic myeloid leukemia (CML), as well as a number of less common types of leukemia.

Diagnosis is typically with blood tests or bone marrow biopsy. Treatment typically includes some combination of chemotherapy, radiation therapy, targeted therapy, and/or bone marrow transplant.

### Mineralocorticoids and Mineralocorticoid Receptors

Mineralocorticoids are a class of corticosteroids produced in the adrenal cortex, and are so named from early observations that these hormones are involved in the retention of sodium, a mineral. Mineralocorticoids bind to the mineralocorticoid receptor in the cell cytosol, and are able to freely cross the lipid bilayer of the cell. Mineralocorticoid receptors become activated upon ligand (e.g., mineralocorticoid) binding. After a hormone binds to the corresponding receptor, the newly formed receptor-ligand complex translocates into the cell nucleus, where it binds to many hormone response elements (HREs) in the promoter region of the target genes in the DNA. By contrast, the hormone receptor without ligand binding also interacts with heat shock proteins and prevents the transcription of targeted genes in a process called transrepression.

The primary endogenous mineralocorticoid is aldosterone; however, other endogenous hormones (e.g., progesterone and deoxycorticosterone) also have mineralocorticoid function. Aldosterone acts on the kidneys to provide active reabsorption of sodium and an associated passive reabsorption of water, as well as the active secretion of potassium in the principal cells of the cortical collecting tubule. Aldosterone is also involved in the active secretion of protons via proton adenosine tri phosphate (ATP)ases in the lumenal membrane of the intercalated cells of the collecting tubule, thereby resulting in an increase of blood pressure and blood volume. The chemical structure of aldosterone is set forth below.

### Aldosterone

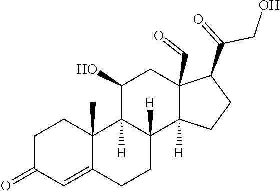

Fludrocortisone is a corticosteroid that acts as a powerful mineralocorticoid, as well as a comparatively weak glucocorticoid. Fludrocortisone, also known as 9α-fluorocortisol (9α-fluorohydrocortisone) or as 9α-fluoro-11β,17α,21-trihydroxypregn-4-ene-3,20-dione, is a synthetic pregnane steroid and a halogenated derivative of cortisol (11β,17α,21-trihydroxypregn-4-ene-3,20-dione). Specifically, fludrocortisone is a modification of cortisol with a fluorine atom substituted in place of one hydrogen atom at the C9a position. The chemical structure of fludrocortisone is set forth below:

### Fludrocortisone

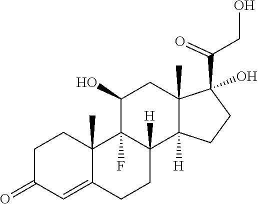

Spironolactone inhibits the effects of mineralocorticoids, namely, aldosterone, by displacing them from the mineralocorticoid receptor in the cortical collecting duct of renal nephrons. This decreases the reabsorption of sodium and water, while limiting the excretion of potassium. Spironolactone has a slightly delayed onset of action, and so it takes several days for diuresis to occur because the MCR is a nuclear receptor which works through regulating gene transcription and gene expression. The chemical structure of spironolactone is set forth below:

### Spironolactone

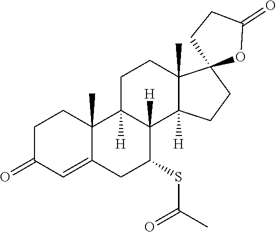

### Inhibition of STAT5 for Cancer Treatment

To better understand the mechanism of STAT5-mediated gene regulation, and to identify strategies to inhibit the function of this protein for cancer therapy, including breast cancer therapy, a chemical biology approach was employed. A cell-based system was developed that allows the quantitative high-throughput measurement of STAT5-dependent gene expression. As described herein, this system was used to interrogate a library of small organic bioactive compounds to identify those that can specifically inhibit STAT5 transcriptional function.

Cross talk between STATs and members of the nuclear receptor family has been observed in normal breast tissue and breast cancer (Cerliani et al., 2011 Cancer Res 71, 3720-3731; Bertucci et al., 2010 Endocrinology 151, 5730-5740; Subtil-Rodriguez et al., 2008 Mol Cell Biol 28, 3830-3849; Carsol et al., 2002 J. Mol Endocrinol 16, 1696-1710; Richer et al., 1998 J Biol Chem 273, 31317-31326; Wyszomierski et al., 1999 Mol Endocrinol 13, 330-343). Progesterone receptor (PR), androgen receptor (AR), and glucocorticoid receptor (GR), have all been shown to synergistically interact with STAT5 and enhance STAT5 target gene expression. As described herein, based on a small molecule screen for STAT5 modulators, it was identified that the MCR also functionally associates with STAT5 and promotes STAT5 transcriptional activity in a subgroup of breast cancer cells.

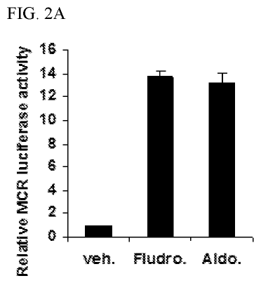

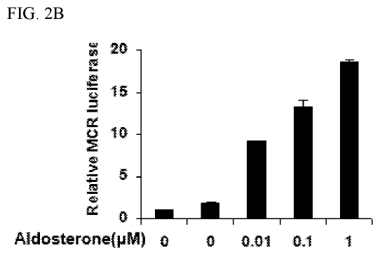

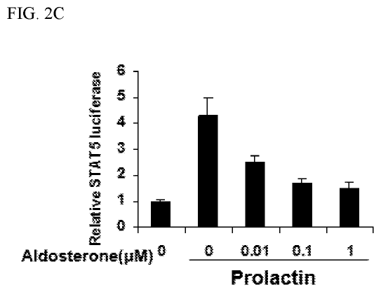

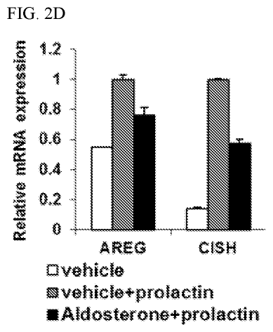

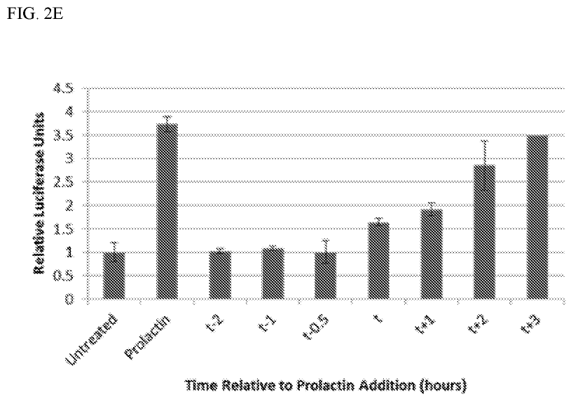

Interestingly, unlike other nuclear receptors where activation by their cognate ligands is required for physical interaction and enhancement of STAT5 transcriptional function, MCR has a distinct interaction with STAT5. It was identified that activation of MCR by its ligands, either the synthetic molecule, fludrocortisone, or the endogenous agonist, aldosterone, paradoxically decreases STAT5 transcriptional function. Indeed, while a dose dependent activation of MCR-dependent transcriptional activity was observed, this corresponded to a dose dependent decrease of STAT5-mediated transcription (FIG. 2B and FIG. 2C). Thus, MCR synergistically interacts with activated STAT5 in the absence of its own ligand, and may be spontaneously recruited to STAT5 or its genomic binding sites upon prolactin-induced STAT5 activation. It is possible that MCR increases the association of STAT5 with transcriptional co-activators such as Brd2, which may be necessary for maximal gene expression (Liu et al., 2014 Molecular cancer therapeutics 13, 1194-1205).

When a physiologic or pharmacologic MCR agonist is present, MCR then binds to its own genomic binding sites and, thus, may be titrated away from its interaction with STAT5, thereby attenuating STAT5 transcriptional function. Alternatively, the presence of an MCR agonist may alter chromatin structure or genomic organization and compromise STAT5 transcriptional function through that mechanism. Nevertheless, using both MCR agonists and genetic approaches, the results presented herein demonstrate a strong functional synergy between MCR and STAT5 in promoting STAT5 target gene expression.

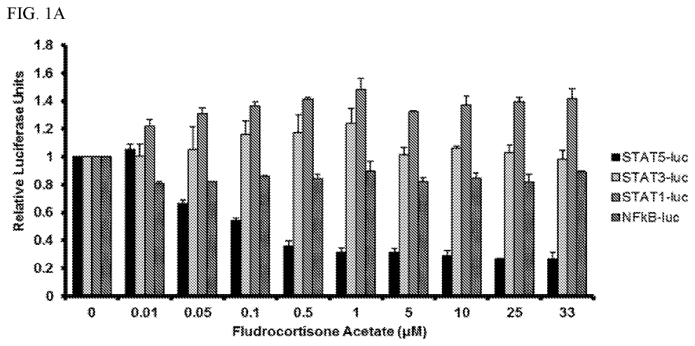

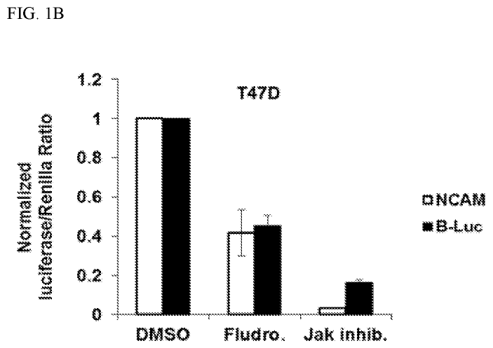

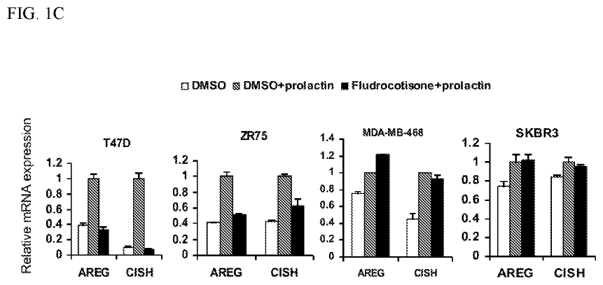

As described in the Examples below, the effect of MCR agonists on STAT5 transcription seems to be highly cell-type specific. For example, fludrocortisone does not appear to inhibit either induced or constitutive STAT5 activity in hematopoietic cells; however, further studies are underway. Also, fludrocortisone seems to only inhibit STAT5 in a subgroup of breast cancer cells. The observed differential effects of MCR agonists on STAT5 transcriptional activity correlates with the expression level of MCR among breast cancer cell lines. Indeed, among the cell lines tested, T47D cells had the highest expression of MCR and were most sensitive to the effects of fludrocortisone in inhibiting STAT5 transcriptional function. In contrast, SKBR3 cells had the lowest expression of MCR among the cell lines tested, and were insensitive to fludrocortisone (FIG. 1C). Thus, the functional modulation of STAT5 by MCR agonists only occur in breast cancer cells with high level of MCR expression.

On the other hand, factors other than MCR expression also influence the functional association between MCR and STAT5. For example, the expression levels of other nuclear receptors in breast cancer cells, which can potentially also interact with STAT5, may have a competitive or synergistic effect on the effects of MCR on STAT5. Adding further to the complexity is that these hormone receptors can clearly display specificity at the level of tissue, cells, and even specific promoters (O'Malley et al., 2009 Cancer Res 69, 8217-8222; Yamamoto et al., 1998 Cold Spring Harb Symp Quant Biol 63, 587-598). Nevertheless, the observation that MCR can modulate STAT5 transcriptional activity in breast cancer cells adds to the spectrum of nuclear receptors that interact with STAT5 and modulate its transcriptional activity.

It is notable that the functional association between STAT5 and MCR arose initially from a chemical biology approach to identifying pharmacological modulators of STAT5 signaling. In addition to identifying therapeutic modulators of transcription factors (Nelson et al., 2008 Blood 112, 5095-5102; Takakura et al., 2011 Human Molecular Genetics 20, 4143-4154; Nelson et al., 2007 Blood 110, 2953; Lynch et al., 2007 Cancer Res 67, 1254-1261), this strategy has been useful for identifying biological probes to uncover basic physiological mechanisms. As described herein, MCR agonists have therapeutic value in the treatment of breast cancer. Elevated circulating levels of prolactin has been reported to be a risk factor for breast cancer (Tworoger et al., 2004 Cancer research 64, 6814-6819; Tworoger et al., 2013 Cancer Research 73, 4810-4819). Furthermore, STAT5 is activated in approximately one-third of primary breast cancers, although this usually occurs in conjunction with activation of STAT3 (Walker et al., 2009 Mol Cancer Res 7, 966-976). Both molecular and epidemiological evidence suggests that the co-activation of STAT5 with STAT3 leads to a less aggressive tumor, perhaps reflecting the role of STAT5 in mediating mammary epithelial differentiation in response to prolactin.

Accordingly, based on findings from a screen of small molecule modulators of STAT5 transcriptional activity described herein, it was identified that unliganded MCR is a co-factor for STAT5 transcriptional activity in breast cancer cells. This finding has both physiologic and therapeutic implications to the functioning of STAT5 in these cells.

As described herein, the compounds of the invention inhibit tumor cell proliferation, inhibit tumor cell number, cell transformation and tumorigenesis in vitro or in vivo using a variety of assays known in the art, or described herein. Such assays can use cells of a cancer cell line or cells from a patient in the presence and absence of the compound of interest. In one example, the cell has dysregulated STAT (e.g., enhanced STAT5 activation). The ability of a compound or a regimen of the invention to reduce the number of cancer cells or inhibit their proliferation can be assessed by, e.g., detecting the expression of antigens on cancer cells or detecting the proliferation of cancer. Techniques known to those of skilled in the art can be used for measuring these activities. For example, cellular proliferation can be assayed by ³H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, flow cytometry and FACS analysis.

### Hyperproliferative Disorders/Neoplasias

The STAT5 inhibitors (i.e., mineralocorticoid receptor agonists and mineralocorticoid receptor antagonists) described herein are useful to treat any hyperproliferative disorder or inflammatory disease driven by increased STAT5 activity. It is contemplated that the methods described herein are particularly useful when the individual has a hyperproliferative disorder characterized by an elevated STAT5 activity, e.g., a neoplasia.

Hyperproliferative disorders include cancerous disease states. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The compounds described herein, e.g., a STAT5 inhibitor, can be used to treat or prevent a variety of hyperproliferative disorders. In some cases, the compounds of the invention are used to treat a cancer with elevated STAT5 activity (e.g., breast cancer and leukemia). For example, the invention is used to treat a solid tumor. In another aspect, the solid tumor is breast cancer, melanoma, colon cancer, ovarian cancer, pancreatic cancer, lung cancer, hepatic cancer, head and neck cancer, prostate cancer and brain cancer. In another example, the hyperproliferative disorder is a hematological cancer such as leukemia or multiple myeloma. Leukemia includes acute lymphoblastic leukemia, acute myelogenous leukemia, chronic myelogenous leukemia, Hodgkin's disease, non-Hodgkin's lymphoma, T-cell lymphoma, B-cell lymphoma and chronic lymphocytic leukemia. The described herein are also used to treat additional hyperproliferative disorders including but not limited to, cancer of the head, neck, eye, skin, mouth, throat, esophagus, chest, bone, lung, colon, sigmoid, rectum, stomach, prostate, ovary, testicle, kidney, liver, pancreas, brain, intestine, heart or adrenals (for a review of such disorders, see Fishman et al., 1985, Medicine, 2d Ed., J.B. Lippincott Co., Philadelphia, incorporated herein by reference).

The medical practitioner can diagnose the patient using any of the conventional cancer screening methods including, but not limited to physical examination (e.g., prostate examination, breast examination, lymph nodes examination, abdominal examination, skin surveillance), visual methods (e.g., colonoscopy, bronchoscopy, endoscopy), PAP smear analyses (cervical cancer), stool guaiac analyses, blood tests (e.g., complete blood count (CBC) test), blood chemistries including liver function tests, prostate specific antigen (PSA) test, carcinoembryonic antigen (CEA) test, cancer antigen (CA)-125 test, alpha-fetoprotein (AFP)), karyotyping analyses, bone marrow analyses (e.g., in cases of hematological malignancies), histology, cytology, a sputum analysis and imaging methods (e.g., computed tomography (CT), magnetic resonance imaging (MRI), ultrasound, X-ray imaging, mammography imaging, bone scans).

### Administration of STAT Inhibitors

Hyperproliferative disorders, including, but not limited to cancer, neoplasms, tumors, metastases, or any disease or disorder characterized by uncontrolled cell growth as known in the art and described herein, can be treated, suppressed, delayed, managed, inhibited or prevented by administering to a subject in need thereof a prophylactically effective regimen or a therapeutically effective regimen, the regimen comprising administering to the patient a compound of the invention, e.g., a STAT5 inhibitor. The invention as it applies to cancer encompasses the treatment, suppression, delaying, management, inhibiting of growth and/or progression, and prevention of cancer or neoplastic disease as described herein.

One aspect of the invention relates to a method of preventing, treating, and/or managing cancer in a patient (e.g., a human patient), the method comprising administering to the patient a prophylactically effective regimen or a therapeutically effective regimen, the regimen comprising administering to the patient a compound of the invention or a composition of the invention, e.g., a STAT5 inhibitor, wherein the patient has been diagnosed with cancer. The amount of a compound of the invention used in the prophylactic and/or therapeutic regimens which will be effective in the prevention, treatment, and/or management of cancer can be based on the currently prescribed dosage of the compound as well as assessed by methods disclosed herein.

In one example, the cancer is a hematologic cancer. For instance, the cancer is leukemia, lymphoma or myeloma. In another example, the cancer is a solid tumor. In some cases, the patient has undergone a primary therapy to reduce the bulk of a solid tumor prior to therapy with the compositions and methods described herein. For example, the primary therapy to reduce the tumor bulk size is a therapy other than a compound or composition of the invention. For example, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, retinoblastoma, embryonal brain tumor, PNET, or choroid plexus tumor. In one aspect, the patient has received or is receiving another therapy. In another aspect, the patient has not previously received a therapy for the prevention, treatment, and/or management of the cancer.

Another aspect of the invention relates to a method of preventing, treating, and/or managing cancer, wherein the patient received another therapy. In some embodiments, the prior therapy is, for example, chemotherapy, radioimmunotherapy, toxin therapy, prodrug-activating enzyme therapy, antibody therapy, surgical therapy, immunotherapy, radiation therapy, targeted therapy or any combination thereof. In some embodiments, the prior therapy has failed in the patient. In some cases, the therapeutically effective regimen comprising administration of a composition of the invention is administered to the patient immediately after patient has undergone the prior therapy. For instance, in certain cases, the outcome of the prior therapy may be unknown before the patient is administered a compound of the invention.

In some cases, the therapeutic regimen results in a reduction in the cancer cell population in the patient. In one example, the patient undergoing the therapeutic regimen is monitored to determine whether the regimen has resulted in a reduction in the cancer cell population in the patient. Typically, the monitoring of the cancer cell population is conducted by detecting the number or amount of cancer cells in a specimen extracted from the patient. Methods of detecting the number or amount of cancer cells in a specimen are known in the art. This monitoring step is typically performed at least 1, 2, 4, 6, 8, 10, 12, 14, 15, 16, 18, 20, or 30 days after the patient begins receiving the regimen.

In one aspect, the specimen may be a blood specimen, wherein the number or amount of cancer cells per unit of volume (e.g., 1 mL) or other measured unit (e.g., per unit field in the case of a histological analysis) is quantitated. The cancer cell population, in certain embodiments, can be determined as a percentage of the total blood cells. In other cases, the specimen extracted from the patient is a tissue specimen (e.g., a biopsy extracted from suspected cancerous tissue), where the number or amount of cancer cells can be measured, for example, on the basis of the number or amount of cancer cells per unit weight of the tissue. The number or amount of cancer cells in the extracted specimen can be compared with the numbers or amounts of cancer cells measured in reference samples to assess the efficacy of the regimen and amelioration of the cancer under therapy. For example, the reference sample is a specimen extracted from the patient undergoing therapy, wherein the specimen from the patient is extracted at an earlier time point (e.g., prior to receiving the regimen, as a baseline reference sample, or at an earlier time point while receiving the therapy). In another example, the reference sample is extracted from a healthy, noncancer-afflicted patient.

In other cases, the cancer cell population in the extracted specimen can be compared with a predetermined reference range. In a specific embodiment, the predetermined reference range is based on the number or amount of cancer cells obtained from a population(s) of patients suffering from the same type of cancer as the patient undergoing the therapy.

### Pharmaceutical Therapeutics

For therapeutic uses, the compositions or agents described herein may be administered systemically, for example, formulated in a pharmaceutically-acceptable buffer such as physiological saline. Preferable routes of administration include, for example, subcutaneous, intravenous, interperitoneal, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

### Formulation of Pharmaceutical Compositions

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

In some cases, the compound or composition of the invention is administered at a dose that is lower than the human equivalent dosage (HED) of the no observed adverse effect level (NOAEL) over a period of three months, four months, six months, nine months, 1 year, 2 years, 3 years, 4 years or more. The NOAEL, as determined in animal studies, is useful in determining the maximum recommended starting dose for human clinical trials. For instance, the NOAELs can be extrapolated to determine human equivalent dosages. Typically, such extrapolations between species are conducted based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs or minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005, incorporated herein by reference.

The amount of a compound of the invention used in the prophylactic and/or therapeutic regimens which will be effective in the prevention, treatment, and/or management of cancer can be based on the currently prescribed dosage of the compound as well as assessed by methods disclosed herein and known in the art. The frequency and dosage will vary also according to factors specific for each patient depending on the specific compounds administered, the severity of the cancerous condition, the route of administration, as well as age, body, weight, response, and the past medical history of the patient. For example, the dosage of a compound of the invention which will be effective in the treatment, prevention, and/or management of cancer can be determined by administering the compound to an animal model such as, e.g., the animal models disclosed herein or known to those skilled in the art. In addition, in vitro assays may optionally be employed to help identify optimal dosage ranges.

In some aspects, the prophylactic and/or therapeutic regimens comprise titrating the dosages administered to the patient so as to achieve a specified measure of therapeutic efficacy. Such measures include a reduction in the cancer cell population in the patient.

In certain cases, the dosage of the compound of the invention in the prophylactic and/or therapeutic regimen is adjusted so as to achieve a reduction in the number or amount of cancer cells found in a test specimen extracted from a patient after undergoing the prophylactic and/or therapeutic regimen, as compared with a reference sample. Here, the reference sample is a specimen extracted from the patient undergoing therapy, wherein the specimen is extracted from the patient at an earlier time point. In one aspect, the reference sample is a specimen extracted from the same patient, prior to receiving the prophylactic and/or therapeutic regimen. For example, the number or amount of cancer cells in the test specimen is at least 2%, 5%, 10%, 15%, 20%, 30%, 40%, 50%, 60%, 70%, 80%, 90%, 95% or 99% lower than in the reference sample.

In some cases, the dosage of the compound of the invention in the prophylactic and/or therapeutic regimen is adjusted so as to achieve a number or amount of cancer cells that falls within a predetermined reference range. In these embodiments, the number or amount of cancer cells in a test specimen is compared with a predetermined reference range.

In other embodiments, the dosage of the compound of the invention in prophylactic and/or therapeutic regimen is adjusted so as to achieve a reduction in the number or amount of cancer cells found in a test specimen extracted from a patient after undergoing the prophylactic and/or therapeutic regimen, as compared with a reference sample, wherein the reference sample is a specimen is extracted from a healthy, noncancer-afflicted patient. For example, the number or amount of cancer cells in the test specimen is at least within 60%, 50%, 40%, 30%, 20%, 15%, 10%, 5%, or 2% of the number or amount of cancer cells in the reference sample.

In treating certain human patients having solid tumors, extracting multiple tissue specimens from a suspected tumor site may prove impracticable. In these cases, the dosage of the compounds of the invention in the prophylactic and/or therapeutic regimen for a human patient is extrapolated from doses in animal models that are effective to reduce the cancer population in those animal models. In the animal models, the prophylactic and/or therapeutic regimens are adjusted so as to achieve a reduction in the number or amount of cancer cells found in a test specimen extracted from an animal after undergoing the prophylactic and/or therapeutic regimen, as compared with a reference sample. The reference sample can be a specimen extracted from the same animal, prior to receiving the prophylactic and/or therapeutic regimen. In specific embodiments, the number or amount of cancer cells in the test specimen is at least 2%, 5%, 10%, 15%, 20%, 30%, 40%, 50% or 60% lower than in the reference sample. The doses effective in reducing the number or amount of cancer cells in the animals can be normalized to body surface area (e.g., mg/m²) to provide an equivalent human dose.

The prophylactic and/or therapeutic regimens disclosed herein comprise administration of compounds of the invention or pharmaceutical compositions thereof to the patient in a single dose or in multiple doses (e.g., 1, 2, 3, 4, 5, 6, 7, 8, 10, 15, 20, or more doses). In one aspect, the prophylactic and/or therapeutic regimens comprise administration of the compounds of the invention or pharmaceutical compositions thereof in multiple doses. When administered in multiple doses, the compounds or pharmaceutical compositions are administered with a frequency and in an amount sufficient to prevent, treat, and/or manage the condition. For example, the frequency of administration ranges from once a day up to about once every eight weeks. In another example, the frequency of administration ranges from about once a week up to about once every six weeks. In another example, the frequency of administration ranges from about once every three weeks up to about once every four weeks.

Generally, the dosage of a compound of the invention administered to a subject to prevent, treat, and/or manage cancer is in the range of 0.01 to 500 mg/kg, e.g., in the range of 0.1 mg/kg to 100 mg/kg, of the subject's body weight. For example, the dosage administered to a subject is in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the patient's body weight. In another example, the dosage of a compound of the invention administered to a subject to prevent, treat, and/or manage cancer in a patient is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a patient's body weight.

In another example, the dosage of a compound of the invention administered to a subject to prevent, treat, and/or manage cancer in a patient is a unit dose of 0.1 to 50 mg, 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In another example, the dosage of a compound of the invention administered to a subject to prevent, treat, and/or manage cancer in a patient is in the range of 0.01 to 10 g/m², and more typically, in the range of 0.1 g/m² to 7.5 g/m², of the subject's body weight. For example, the dosage administered to a subject is in the range of 0.5 g/m² to 5 g/m², or 1 g/m² to 5 g/m² of the subject's body's surface area.

In another example, the prophylactic and/or therapeutic regimen comprises administering to a patient one or more doses of an effective amount of a compound of the invention, wherein the dose of an effective amount achieves a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention.

In another example, the prophylactic and/or therapeutic regimen comprises administering to a patient a plurality of doses of an effective amount of a compound of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention for at least 1 day, 1 month, 2 months, 3 months, 4 months, 5 months, 6 months, 7 months, 8 months, 9 months, 10 months, 11 months, 12 months, 15 months, 18 months, 24 months or 36 months.

In other embodiments, the prophylactic and/or therapeutic regimen comprises administering to a patient a plurality of doses of an effective amount of a compound of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention for at least 1 day, 1 month, 2 months, 3 months, 4 months, 5 months, 6 months, 7 months, 8 months, 9 months, 10 months, 11 months, 12 months, 15 months, 18 months, 24 months or 36 months.

### Combination Therapy

In one example, the active compounds are administered in combination therapy, i.e., combined with other agents, e.g., therapeutic agents, that are useful for treating pathological conditions or disorders, such as various forms of cancer. The term “in combination” in this context means that the agents are given substantially contemporaneously, either simultaneously or sequentially. If given sequentially, at the onset of administration of the second compound, the first of the two compounds is in some cases still detectable at effective concentrations at the site of treatment.

The administration of a compound or a combination of compounds for the treatment of a neoplasia may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Accordingly, in some examples, the prophylactic and/or therapeutic regimen comprises administration of a compound of the invention in combination with one or more additional anticancer therapeutics. In one example, the dosages of the one or more additional anticancer therapeutics used in the combination therapy is lower than those which have been or are currently being used to prevent, treat, and/or manage cancer. The recommended dosages of the one or more additional anticancer therapeutics currently used for the prevention, treatment, and/or management of cancer can be obtained from any reference in the art including, but not limited to, Hardman et al., eds., Goodman & Gilman's The Pharmacological Basis Of Basis Of Therapeutics, 10th ed., McGraw-Hill, New York, 2001; Physician's Desk Reference (60^{th} ed., 2006), which is incorporated herein by reference in its entirety.

The compound of the invention and the one or more additional anticancer therapeutics can be administered separately, simultaneously, or sequentially. In various aspects, the compound of the invention and the additional anticancer therapeutic are administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In another example, two or more anticancer therapeutics are administered within the same patient visit.

In certain aspects, the compound of the invention and the additional anticancer therapeutic are cyclically administered. Cycling therapy involves the administration of one anticancer therapeutic for a period of time, followed by the administration of a second anticancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In another example, the anticancer therapeutics are administered concurrently to a subject in separate compositions. The combination anticancer therapeutics of the invention may be administered to a subject by the same or different routes of administration.

When a compound of the invention and the additional anticancer therapeutic are administered to a subject concurrently, the term “concurrently” is not limited to the administration of the anticancer therapeutics at exactly the same time, but rather, it is meant that they are administered to a subject in a sequence and within a time interval such that they can act together (e.g., synergistically to provide an increased benefit than if they were administered otherwise). For example, the anticancer therapeutics may be administered at the same time or sequentially in any order at different points in time; however, if not administered at the same time, they should be administered sufficiently close in time so as to provide the desired therapeutic effect, preferably in a synergistic fashion. The combination anticancer therapeutics of the invention can be administered separately, in any appropriate form and by any suitable route. When the components of the combination anticancer therapeutics are not administered in the same pharmaceutical composition, it is understood that they can be administered in any order to a subject in need thereof. For example, a compound of the invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional anticancer therapeutic, to a subject in need thereof. In various aspects, the anticancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the anticancer therapeutics are administered within the same office visit. In another example, the combination anticancer therapeutics of the invention are administered at 1 minute to 24 hours apart.

### Release of Pharmaceutical Compositions

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level. Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Kits or Pharmaceutical Systems

The present compositions may be assembled into kits or pharmaceutical systems for use in ameliorating a neoplasia. Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### Mineralocorticoids as Inhibitors of STAT5

As described herein, the transcription factor STAT5 regulates gene expression triggered by hormones (including growth hormone and prolactin) and cytokines (like interleukin-2 (IL-2) and IL-3). STAT5-regulated genes control critical cellular processes such as proliferation, survival, and differentiation. Constitutive STAT5 function occurs in many human tumors, including leukemias and breast cancer. Furthermore, as a mediator of the immune-stimulating cytokine IL-2, STAT5 activity is elevated in many inflammatory and auto-immune conditions.

As described in detail below, to identify compounds that can inhibit STAT5, a cell line in which STAT5 activity leads to the expression of the light-emitting enzyme, luciferase, was developed. Then, these cells were used to screen a library of bioactive compounds to identify those that specifically inhibit STAT5 transcriptional function. As described herein, the FDA-approved mineralocorticoid agonist fludrocortisone is a potent and specific inhibitor of STAT5. Furthermore, the endogenous mineralocorticoid aldosterone and the pharmacologic antagonist, spironolactone, also inhibit STAT5 function. As described herein, these compounds work by titrating away the mineralocorticoid receptor, which serves as a co-activator, from STAT5. The findings presented herein suggest that fludrocortisone and related molecules have important anti-cancer and immunomodulatory activities.

Transcription factors have often been viewed as “undruggable” targets because they lack structures that would make them susceptible to inhibition by small organic molecules. Nonetheless, STAT5 is a very appealing target. While it can be inhibited in normal cells with little toxicity, many tumor types depend on continual STAT5 activation for their survival. In addition, activated immune cells also rely on STAT5 for proliferation. However, prior to the invention described herein, a drug to inhibit STAT5 directly had not been developed. As described in the Examples below, the finding that fludrocortisone (and related compounds) block STAT5 function specifically, at nanomolar concentrations, raises the possibility of targeting STAT5 directly in a number of important disease states. The results presented herein demonstrate that fludrocortisone (and/or related compounds) can be used therapeutically in cancer and inflammatory diseases driven by increased STAT5 activity, both alone and in combination.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Materials and Methods

### Cell Lines and Stimulations

T47D, ZR75 (American Type Culture Collection, Manassas Va.), and MDA-MB-468, were maintained in DMEM containing 10% FBS. SK-BR-3 cells were maintained in RPMI 1640 with 10% FBS. Cells were stimulated with 100 ng/mL prolactin or 10 ng/ml IL6 (R&D Systems, Minneapolis, Minn.). STAT1, STAT3, STAT5 and NF-κB-dependent luciferase activities were measured in STAT-luc/2FTGH, STAT-luc/U3A, NCAM2-luc/T47D and NF-κB-luc/293 cells, respectively (Nelson et al., 2008 Blood 112, 5095-5102). Luciferase activity was quantified using the Bright-Glo Luciferase assay system (Promega, Madison, Wis.).

### Compounds

The Prestwick Chemical Library was obtained from Prestwick Chemicals (Illkirch, France). Fludrocortisone acetate and aldosterone were purchased from Sigma. Jak Inhibitor 1 was obtained from EMD Millipore (Billerica, Mass.). Fludrocortisone acetate and JAK inhibitor 1 were dissolved in DMSO and were diluted to a final concentration of 0.1% DMSO in all experiments. Aldosterone was dissolved in 100% ethanol.

### RNA Interference

siRNAs targeting MCR (sc-38836) and scrambled controls were obtained from Santa Cruz Biotechnology (Dallas, Tex.) and transfected into cells using Lipofectamine RNAiMAX (Invitrogen, Carlsbad, Calif.).

### Dual Luciferase Assays

Cells were transfected with plasmids expressing firefly luciferase under the control of STAT5-responsive regulatory regions from the NCAM2 gene (NCAM-luc or NCAM2-luciferase; Nelson et al., 2006 J Biol Chem 281, 26216-26224, incorporated herein by reference) or region B of the BCL6 gene (B-luc or B-luciferase; Walker et al., 2007 Oncogene 26, 224-233, incorporated herein by reference). Cells were also co-transfected with a plasmid expressing Renilla luciferase under a constitutive promoter for normalization (pRL-TK plasmid; Promega; Madison, Wis., incorporated herein by reference). After 48 hours fludrocortisone was added, and then one hour later cells were left unstimulated or treated with prolactin. Five hours after cytokine stimulation, cells were harvested for dual luciferase assay (Promega). For ectopic expression of MCR, cells were also co-transfected with either an empty vector control or a vector expressing MCR (pCMX-MCR; Mueller et al, 2014, Endocrinology, 155(11): 4461-4472, incorporated herein by reference). Cytokine stimulation was performed 48 hours after plasmid transfection.

### Immunoblots and Chromatin Immunoprecipitation (ChIP)

Nuclear and cytoplasmic fractionation was performed using the Nuclear Extract Kit (Active Motif, Carlsbad, Calif.). Immunoblots were performed using antibodies recognizing STAT5 (sc-482), MCR (sc-11412) and RNA polymerase II (pol II) (sc-9001) (all from Santa Cruz Biotechnology, Santa Cruz Calif.) and tyrosine-phosphorylated STAT5 (9131L) (from Cell Signaling Technology, Danvers, Mass.).

ChIP was performed as described (Nelson et al., 2004 J Biol Chem 279, 54724-54730). Briefly, T47D cells were fixed with formaldehyde then sonicated, and lysates were immunoprecipitated with antibodies to STAT5 or RNA polymerase II (pol II). Quantitative PCR was performed in triplicate on ChIP product or input using SYBR Green PCR Master Mix (Applied Biosystems, Foster City, Calif.) and region-specific primers as follows: CISH, forward, 5′-CCCGCGGTTCTAGGAAGAC-3′ (SEQ ID NO: 1), reverse, 5′-CGAGCTGCTGCCTAATCCT-3′ (SEQ ID NO: 2); Rhodopsin, forward, 5′-TGGGTGGTGTCATCTGGTAA-3′ (SEQ ID NO: 3), reverse, 5′-GGATGGAATGGATCAGATGG-3′ (SEQ ID NO: 4). Relative binding at the CISH promoter site was normalized to input and expressed as mean fold change relative to binding at the Rhodopsin site, which is a control DNA binding site known not to bind STAT5.

### Gene Expression Analysis

RNA was isolated using the RNeasy kit (QIAGEN, Valencia, Calif.). cDNA was generated using the TaqMan reverse transcription kit (Applied Biosystems, Foster City, Calif.). Quantitative real-time PCR was performed in triplicate using SYBR green master mix (Applied Biosystems) on a model 7500 real time PCR system (Applied Biosystems). Data are expressed as mean fold change±standard deviation of three replicates. The following primers were used: AREG, forward, 5′-TGGTGCTGTCGCTCTTGATA-3′ (SEQ ID NO: 5), reverse, 5′-TCACGCTTCCCAGAGTAGGT-3′ (SEQ ID NO: 6); CISH, forward, 5′-CTGCTGTGCATAGCCAAGAC-3′ (SEQ ID NO: 7), reverse, 5′-GTGCCTTCTGGCATCTTCTG-3′ (SEQ ID NO: 8); and, 18s, forward, 5′-GTAACCCGTTGAACCCCATT-3′ (SEQ ID NO: 9), reverse, 5′-CCATCCAATCGGTAGTAGCG-3′ (SEQ ID NO: 10).

### Example 2: The Synthetic Mineralocorticoid Fludrocortisone Inhibits STAT5 Transcriptional Activity in Breast Cancer Cells

To identify small molecules that modulate STAT5 transcriptional activity, T47D cells stably expressing a luciferase reporter gene under the control of a STAT5-dependent promoter were used. Basal luciferase expression is minimal in these cells. However, when the cells are treated with prolactin (100 ng/ml), robust and reproducible luciferase expression can be detected. Using this system, the Prestwick Chemical Library, which is biased towards bioactives, was screened to identify compounds that could specifically modulate STAT5 transcriptional function. Through this approach, fludrocortisone was identified as a potent and specific inhibitor of STAT5 (FIG. 1A). Fludrocortisone inhibited STAT5-dependent luciferase activity, with an IC₅₀ of approximately 0.1 μM. By contrast, at concentrations up to 33 μM, fludrocortisone had essentially no effect on NF-κB, STAT1 or STAT3-dependent luciferase expression.

To exclude the possibility that this effect of fludrocortisone was limited to this single reporter system, parental T47D cells transfected with a reporter construct in which luciferase is regulated by one of two distinct STAT5-dependent promoter sequences derived from the NCAM2 gene (NCAM-luc) (Nelson et al., 2006 J Biol Chem 281, 26216-26224) or the BCL6 gene (B-luc) (Walker et al., 2007 Oncogene 26, 224-233) were used. Fludrocortisone reduced STAT5-dependent luciferase activity mediated by both of these promoters in T47D cells to an equivalent extent (FIG. 1B), suggesting that this effect was not limited to a single promoter sequence.

### Example 3: Fludrocortisone Inhibits Expression of Endogenous STAT5 Target Genes in a Subgroup of Breast Cancer Cells

Having identified that fludrocortisone inhibits the expression of STAT5-dependent reporter constructs, the expression of endogenous STAT5 target genes in multiple breast cancer cell lines was analyzed. It was identified that fludrocortisone effectively inhibits expression of the endogenous STAT5 target genes amphiregulin (AREG) and cytokine inducible SH2-containing protein (CISH) in T47D and ZR75 cells, but not in SKBR3 and MDA-MB-468 cells (FIG. 1C). These findings indicated that while fludrocortisone can inhibit endogenous STAT5 transcriptional activity, it does not do so in all cell lines.

### Example 4: The Endogenous MCR Ligand Aldosterone Inhibits STAT5 Transcriptional Activity in Breast Cancer Cells

Since fludrocortisone is a synthetic corticosteroid with mainly mineralocorticoid activity, it was next examined whether the endogenous mineralocorticoid receptor agonist aldosterone has a similar effect. It was identified that both fludrocortisone and aldosterone activate MCR in breast cancer cells, as evaluated by MCR-specific luciferase reporter (FIG. 2A), and aldosterone activates MCR in a dose-dependent manner (FIG. 2B). Similar to fludrocortisone, aldosterone showed a dose-dependent inhibition of a STAT5-dependent reporter gene (FIG. 2C). Furthermore, aldosterone inhibited endogenous expression of bona fide endogenous STAT5 target genes (FIG. 2D). Thus, this STAT5 inhibitory effect is not unique to fludrocortisone, but is shared by a principal endogenous MCR agonist as well.

### Example 5: The Inhibition of STAT5 Function by Fludrocortisone is a Rapid Effect

To further characterize the inhibition of STAT5 function by fludrocortisone, the temporal relationship between fludrocortisone addition and inhibition of prolactin-induced STAT5-dependent gene expression was analyzed. It was identified that even when fludrocortisone was added simultaneously with prolactin, it can still effectively inhibit prolactin-stimulated STAT5 luciferase activity, and still mediated some inhibitory effect when added after prolactin (FIG. 2E). These results indicate that the inhibitory effects of fludrocortisone on STAT5 transcriptional activity occur rapidly, suggesting that it is a direct genomic effect that is not dependent on subsequent gene expression and protein synthesis.

### Example 6: Fludrocortisone does not Affect STAT5 Phosphorylation or DNA Binding

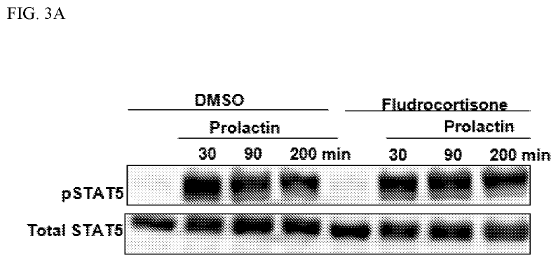

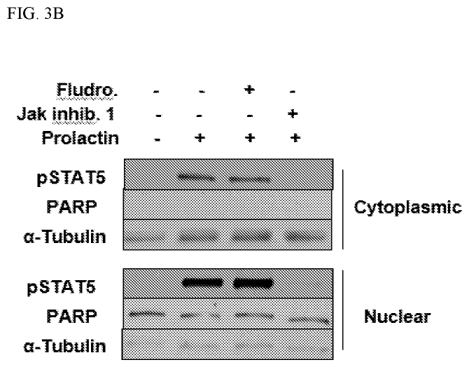

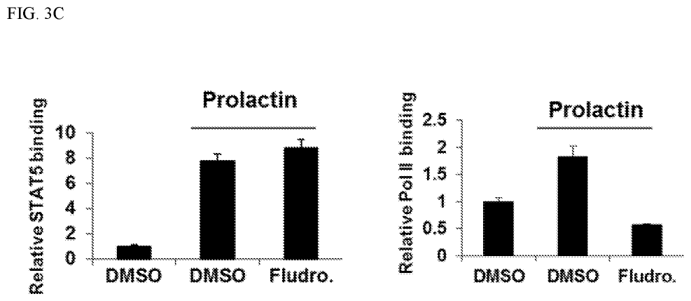

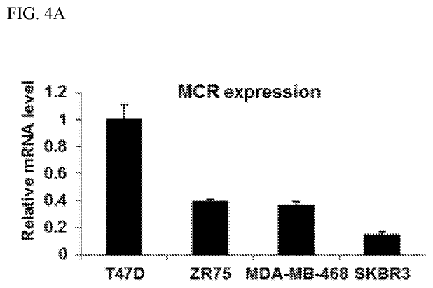

Next, it was considered whether fludrocortisone was inhibiting STAT5 function by blocking a key step in its activation. To address this, the effect of fludrocortisone on the kinetics of STAT5 phosphorylation following prolactin stimulation was evaluated. However, no difference in the dynamics of STAT5 activation in the presence of fludrocortisone was identified (FIG. 3A). It was then evaluated whether nuclear translocation of activated STAT5 was affected by fludrocortisone treatment. T47D cells were either untreated or pretreated with fludrocortisone or JAK inhibitor 1, then left unstimulated or treated with prolactin, after which nuclear and cytoplasmic fractions were separated and analyzed for phosphorylated STAT5. Fludrocortisone had no effect on the phosphorylation or nuclear localization of STAT5 (FIG. 3B). By contrast, pre-treatment with the Jak inhibitor led to complete inhibition of phosphorylation and nuclear localization of STAT5. Thus, the inhibitory effect of fludrocortisone must be occurring within the nucleus. Next, the possibility that fludrocortisone is inhibiting the interaction of STAT5 with its cognate genomic regulatory sequences was considered. To examine this, chromatin immunoprecipitation (ChIP) was performed to evaluate the effect of fludrocortisone on the binding of STAT5 to its well-characterized regulatory sites in CISH. Following stimulation with prolactin, STAT5 binding to this site increased by approximately 8 fold. However, fludrocortisone had no effect on STAT5 binding to this element. By contrast, consistent with its inhibition of transcription of STAT5 target genes, fludrocortisone did inhibit the prolactin-induced recruitment of RNA polymerase II (pol II) to this promoter site (FIG. 3C).

### Example 7: Fludrocortisone Inhibits STAT5 Transcription Through the MCR

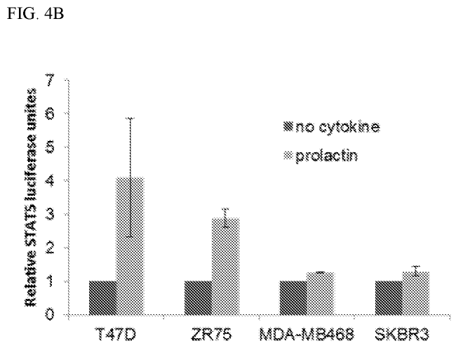

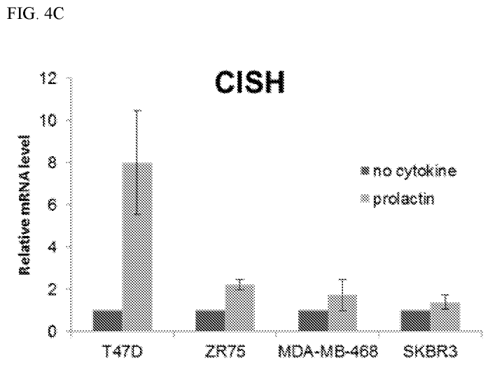

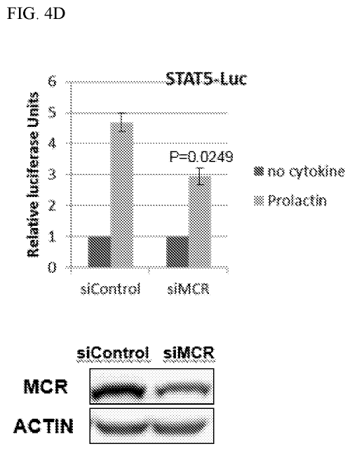

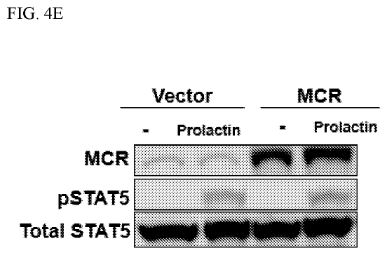

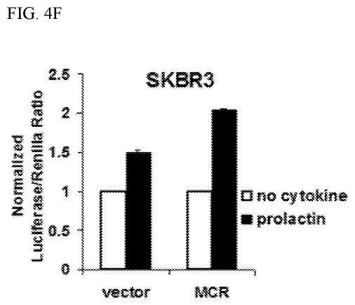

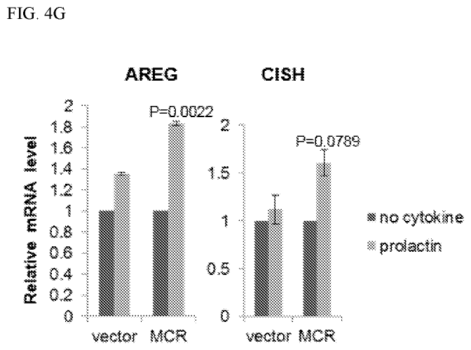

As MCR is the immediate biological target of fludrocortisone (and aldosterone), the hypothesis that MCR played a direct role in fludrocortisone-mediated STAT5 inhibition was examined. Since it was identified that fludrocortisone only inhibits STAT5 activity in a subset of breast cancer cells (FIG. 1C), it was first evaluated whether differences in MCR expression among the four breast cancer cell lines could account for these differences. Interestingly, it was identified that cells lines in which fludrocortisone inhibited STAT5 activity (T47D and ZR75) displayed higher levels of MCR expression than cell lines (MDA-MB-468 and SKBR3) in which STAT5 function was not inhibited by fludrocortisone (FIG. 4A). Furthermore, prolactin-induced STAT5 transcriptional activity, as measured by both luciferase reporters and endogenous target genes expression, was higher in T47D and ZR75 than MDA-MB-468 and SKBR3 cells (FIG. 4B and FIG. 4C). These observations indicate that MCR expression correlates with STAT5 transcriptional activity in breast cancer cells.

To further evaluate the role of MCR in STAT5 transcriptional activity, RNA interference was used to inhibit MCR expression in T47D cells, which had the highest level of MCR expression among the breast cancer cell lines tested. Reduction of MCR led to a 50% reduction of prolactin-induced STAT5 transcriptional activity (FIG. 4D). Then, MCR was over-expressed in SKBR3 cells, which have low levels of MCR expression. While changes in MCR expression had no effect on prolactin-induced STAT5 phosphorylation (FIG. 4E), increased expression of MCR led to an increase in STAT5 transcriptional activity, as evaluated by both luciferase reporter constructs and expression of endogenous STAT5 target genes (FIG. 4F and FIG. 4G). Taken together, these data indicate that MCR promotes STAT5 transcriptional activity in breast cancer cells with high level of MCR expression.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of inhibiting signal transducer and activator of transcription 5 (STAT5) function or activity in a cell comprising contacting the cell with a mineralocorticoid receptor agonist or a mineralocorticoid receptor antagonist, or an analogue thereof, thereby inhibiting STAT5 function or activity in a cell.

2. The method of claim 1, wherein the mineralocorticoid receptor agonist comprises aldosterone, fludrocortisone, desoxycortone, hydrocortisone, methylprednisolone, prednisolone, prednisone, or an analogue thereof.

3. The method of claim 1, wherein the mineralocorticoid receptor antagonist comprises spironolactone, canrenoate potassium, canrenone, drospirenone, dydrogesterone, eplerenone, gestodene, medrogestone, progesterone, trimegestone, amlodipine, aspararenone, benidipine, esaxerenone, felodipine, finerenone, nifedipine, nimodipine, nitrendipine, or an analogue thereof.

4. The method of claim 1, wherein the STAT5 function or activity comprises STAT5-dependent gene expression/transcriptional activity.

5. The method of claim 1, wherein the mineralocorticoid receptor agonist or the mineralocorticoid receptor antagonist inhibits expression of a STAT5 target gene selected from the group consisting of amphiregulin (AREG), cytokine inducible SH2-containing protein (CISH), B-cell lymphoma 2 (Bcl-2), B-cell lymphoma-extra large (Bcl-x1), suppressor of cytokine signaling 1 (SOCS1), SOCS3, oncostatin-M, mitogen-activated protein (MAP) kinase phosphatase-1 (MKP-1), Pim-1, Pim-2, p21 (CIP/WAF1), interleukin-2 receptor a (IL-2Ra), IL-2Rβ, related to receptor tyrosine kinase (Ryk), and tumor necrosis factor receptor superfamily member 13B (TNFRSF13b).

6. The method of claim 1, wherein the mineralocorticoid receptor agonist or the mineralocorticoid receptor antagonist is administered at a dose of 0.01 μM to 10 μM.

7. The method of claim 1, wherein STAT5 function or activity in the cell is inhibited by 10%-100%.

8. A method for treating or preventing cancer or an inflammatory disease associated with aberrant STAT5 function or activity in a human subject comprising:
administering to the subject a therapeutically effective amount of a mineralocorticoid receptor agonist or a mineralocorticoid receptor antagonist, or an analogue thereof, thereby treating or preventing the cancer or inflammatory disease associated with aberrant STAT5 function or activity in the human subject

9. (canceled)

10. The method of claim 8, wherein the human subject is identified as having elevated STAT5 function or activity, or wherein the human subject is identified as in need of inhibiting STAT5 function or activity, or wherein the human subject has been diagnosed with a cancer or an inflammatory disease associated with aberrant STAT5 activity.

11. The method of claim 8, wherein the mineralocorticoid receptor agonist comprises aldosterone, fludrocortisone, desoxycortone, hydrocortisone, methylprednisolone, prednisolone, prednisone, or an analogue thereof.

12. The method of claim 8, wherein the mineralocorticoid receptor antagonist comprises spironolactone, canrenoate potassium, canrenone, drospirenone, dydrogesterone, eplerenone, gestodene, medrogestone, progesterone, trimegestone, amlodipine, aspararenone, benidipine, esaxerenone, felodipine, finerenone, nifedipine, nimodipine, nitrendipine, or an analogue thereof.

13. The method of claim 8, wherein the STAT5 function or activity comprises STAT5-dependent gene expression/transcriptional activity.

14. The method of claim 8, wherein the mineralocorticoid receptor agonist or the mineralocorticoid receptor antagonist inhibits expression of a STAT5 target gene selected from the group consisting of AREG, CISH, Bcl-2, Bcl-x1, SOCS1, SOCS3, oncostatin-M, MKP-1, Pim-1, Pim-2, p21 (CIP/WAF1), IL-2Rα, IL-2Rβ, Ryk, and TNFRSF13b.

15. The method of claim 8, wherein the mineralocorticoid receptor agonist or the mineralocorticoid receptor antagonist is administered at a dose of 0.01 μM to 10 μM.

16. (canceled)

17. (canceled)

18. The method of claim 8, wherein the cancer comprises a solid tumor selected from the group consisting of breast cancer, melanoma, colon cancer, ovarian cancer, pancreatic cancer, lung cancer, hepatic cancer, head and neck cancer, prostate cancer and brain cancer.

19. The method of claim 8, wherein the cancer comprises leukemia selected from the group consisting of acute lymphoblastic leukemia, acute myelogenous leukemia, chronic myelogenous leukemia, Hodgkin's disease, non-Hodgkin's lymphoma, T-cell lymphoma, B-cell lymphoma and chronic lymphocytic leukemia or multiple myeloma.

20. (canceled)

21. The method of claim 8, wherein the inflammatory disease associated with aberrant STAT5 activity comprises systemic lupus erythematosus, multiple sclerosis, Crohn's disease, ulcerative colitis, or graft versus host disease.

22. (canceled)

23. The method of claim 8, further comprising administering a chemotherapeutic agent selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and vinorelbine.

24. An isolated T47D breast cancer cell comprising a vector expressing a firefly luciferase reporter gene operably-linked to a STAT5-dependent promoter.

25. (canceled)

26. The isolated breast cancer cell of claim 24, wherein the STAT5-dependent promoter comprises a sequence derived from a neural cell adhesion molecule 2 (NCAM2) gene or region B of a B-cell lymphoma 6 (BCL6) gene.

27. The isolated breast cancer cell of claim 26, wherein the cell comprises a vector expressing Renilla luciferase operably linked to a constitutive promoter.

28. The isolated breast cancer cell of claim 27, wherein the cell comprises a vector expressing MCR.

29. A method of screening for a compound that inhibits STAT5 function and/or activity comprising:
providing one or more breast cancer cell(s) comprising a vector expressing a firefly luciferase reporter gene operably-linked to a STAT5-dependent promoter;
and contacting the cell(s) with a candidate compound, wherein a decrease in the level of STAT5-dependent luciferase activity in the presence of the candidate compound as compared to the level of STAT5-dependent luciferase activity in the absence of the candidate compound indicates that the candidate compound inhibits STAT5 function and/or activity.

30. The method of claim 29, further comprising contacting the cell with an agent that induces the function and/or activity of STAT5 prior to contacting the cell with a candidate compound.

31. The method of claim 30, wherein the agent that induces the function and/or activity of STAT5 comprises prolactin.


SEQUENCE LISTING (14 sequences)

SEQ ID NO: 1 - DNA, 19 bp, Artificial Sequence
  Chemically synthesized primer
cccgcggttctaggaagac

SEQ ID NO: 2 - DNA, 19 bp, Artificial Sequence
  Chemically synthesized primer
cgagctgctgcctaatcct

SEQ ID NO: 3 - DNA, 20 bp, Artificial Sequence
  Chemically synthesized primer
tgggtggtgtcatctggtaa

SEQ ID NO: 4 - DNA, 20 bp, Artificial Sequence
  Chemically synthesized primer
ggatggaatggatcagatgg

SEQ ID NO: 5 - DNA, 20 bp, Artificial Sequence
  Chemically synthesized primer
tggtgctgtcgctcttgata

SEQ ID NO: 6 - DNA, 20 bp, Artificial Sequence
  Chemically synthesized primer
tcacgcttcccagagtaggt

SEQ ID NO: 7 - DNA, 20 bp, Artificial Sequence
  Chemically synthesized primer
ctgctgtgcatagccaagac

SEQ ID NO: 8 - DNA, 20 bp, Artificial Sequence
  Chemically synthesized primer
gtgccttctggcatcttctg

SEQ ID NO: 9 - DNA, 20 bp, Artificial Sequence
  Chemically synthesized primer
gtaacccgttgaaccccatt

SEQ ID NO: 10 - DNA, 20 bp, Artificial Sequence
  Chemically synthesized primer
ccatccaatcggtagtagcg

SEQ ID NO: 11 - protein, 794 aa, Homo sapiens
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 12 - DNA, 4414 bp, Homo sapiens
agatggccggagtaaaagaaggagggaggtgctgcggtggtgggggtgatcttggcttcactagaatccccagttcttcccctctctacagttttgtctctgaggtcacaaaacctgtggcccccaagacacacatgcgcacacacgcgcgtgcacacacacaccccacacatttattttttaatctaggggctcaaaagatgacacgcgccagagctggaaggcgtcgccaattggtccacttttccctcctccctttttgcggatgagaaaactgaggcccaggtttgggatttccagagcccgggatttcccggcaacgcccgacaaccacattcccccggctattctgacccgccccggttccgggacgctccctgggagccgccgccgagggcctgctgggactcccgggggaccccgccgtcggggcagcccccacgcccggcgccgcccgccgggaacggccgccgctgttgcgcacttgcaggggagccggcgactgagggcgaggcagggagggagcaagcggggctgggagggctgctggcgcgggctcgcgcgctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggctgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcataaaaaaaaaaaaaaaa

SEQ ID NO: 13 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 14 - DNA, 5171 bp, Homo sapiens
ggcgggaggagagtcggcggccggagccgtcaccccgggcggggacccagcgcaggcaactccgcgcggcggcccggccgagggagggagcgagcgggcgggcgggcaagccagacagctgggccggagcagccgcgggcgcccgaggggccgagcgagattgtaaaccatggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcaccattgcttggaagtttgattctcaggaaagaatgttttggaatctgatgccttttaccaccagagacttctccattcggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgtgaccccgcgacctctccatcttcagcttcttcatcttcaccagaggaatcactcttgtggatgttttaattccatgaatcgcttctcttttgaaacaatactcataatgtgaagtgttaatactagttgtgaccttagtgtttctgtgcatggtggcaccagcgaagggagtgcgagtatgtgtttgtgtgtgtgtgtgtgtgtgtgtgtgtgtgcgtgtttgcacgttatggtgtttctccctctcactgtctgagagtttagttgtagcagaggggccacagacagaagctgtggtggtttttactttgtgcaaaaaggcagtgagtttcgtgaagcctggaagttggccatgtgtcttaagagtggctggactttgacatgtggctgtttgaataagagaaggacaaagggaggagaaagcacatgtgctccagtgagtcttcgtcactctgtctgccaagcaattgatatataaccgtgattgtctctgcttttcttctgaaatgtagataactgctttttgacaaagagagccttccctctcccccacccctgtgttcttgggtaggaatgggaaaaggggcaacctacaaagattgttggggcaagggaagtcacaagctttcggatgggcggtggcttttcacaaaacatttagctcatcttattctctctttgtcctctctcccctcctgcccgcccgcaccctggaattgccactcagttcctctgggtgtgcacatatgtttggagaaatagaggagagaaaagagggccacgtaactgagagcttacagtgccaatgccgtttgtgttctggccagagtggagtgcgcagccctgactcccaggcgctgagattgttgcctggttacccaggaagctgctgttccggctgcccagcctttctctgagccagcggatgcacagtccgtggccttcttcaggcttattgatgatgctttttgcaaatgttgaatcatggttctgtttctaagttggatcttttttgttttctccttgccaccctaatttgacatcaaaattctctcttgtgcattgggccctgggtcattcaaacccaggtcacctcattccccttctctgttcacacctaatgtcttgaagagtaggtagcagcagtgtgggctgaacctaggccagcttgcttagcgggtcaccctgctgtgaagtcctggcaggtgttggtaatgtgtggaaatgcagtcagcaagtttgctggggagtttgataaaagtataaaacaaaacaaaaaaagcctcggtataattttgttccacgacttcttctgtagctttacaccagaaggaaggaatgggctacagcaggtagtggaggaagaggggggtgagcaggtgtattaaaatagcttacgggtaaggcctaaaaggtcacccctcggccccctctccaaaagaagggcatgggcacccccaggagaggatggccccaaaaaccttatttttatacatgagagtaaataaacatattttttttacaaaaataacttctgaatttatcagtgttttgccgttaaaaatattcctctatagtaaattatttattggaagatgacttttttaaagctgccgtttgccttggcttggtttcatacactgatttatttttctatgccaggcagtagagtctctctgcctctgaggagcaggctacccgcatcccactcagcccctccctacccctcaagatttgatgaaaattccaaccatgaggatgggtgcatcggggaagggtgagaaggagagcctgcctgctcagggatccaggctcgtagagtcactccctgcccgtctcccagagatgcttcaccagcacctgcctctgagacctcgctctctgttccagcaaccctggttggggggtcagacttgatacactttcaggttgggagtggacccaccccagggcctgctgaggacagagcagccaggccgtcctggctcactttgcagttggcactgggttggggaggaagagagctgatgagtgtggcttccctgagctggggtttccctgcttgtccagttgtgagctgtcctcggtgttaccgaggctgtgcctagagagtggagatttttgatgaaaggtgtgctcgctctctgcgttctatcttctctctcctccttgttcctgcaaaccacaagataaaggtagtggtgtgtctcgaccccatcagcctctcacccactcccagacacacacaagtcctcaaaagtttcagctccgtgtgtgagatgtgcaggttttttctagggggtagggggagactaaaatcgaatataacttaaaatgaaagtatactttttataatttttctttttaaaacttggtgaaattatttcagatacatattttagtgtcaaggcagattagttatttagccaccaaaaaaaagtattgtgtacaatttggggcctcaaatttgactctgcctcaaaaaaaagaaatatatcctatgcagagttacagtcacaaagttgtgtattttatgttacaataaagccttcctctgaagggaaaaaaaaaaaaaaaaaaaaaaaaaaaaaaaaaaaaaaaaaaaaaaaaaaaaaaaaaaaaaaaaaaaaaaaaaaaaaa